(19) United States
(12) Patent Application Publication
(10) Pub. No.: US 2016/0069888 A1
(43) Pub. Date: Mar. 10, 2016

(54) METHOD FOR THE DIAGNOSTIC OF CANCER AND ENZYME-LINKED IMMUNOASSAY (ELISA) KIT FOR ITS APPLICATION

(71) Applicants: Evgeny Iosifovich GOUFMAN, Moscow (RU); Vasily Nikolaevich YAKOVLEV, Moscow (RU); Alexei Alexeevich KANAEV, Moscow (RU); Rustam Raisovich SULEIMANOV, Moscow (RU)

(72) Inventors: Evgeny Iosifovich GOUFMAN, Moscow (RU); Vasily Nikolaevich YAKOVLEV, mOSCOW (RU); Alexei Alexeevich KANAEV, Moscow (RU); Rustam Raisovich SULEIMANOV, Moscow (RU)

(21) Appl. No.: 14/442,130

(22) PCT Filed: Dec. 29, 2012
(86) PCT No.: PCT/RU2012/001148

§ 371 Date: May 12, 2015

(30) Foreign Application Priority Data
Nov. 14, 2012 (RU) 2012148244

(51) Int. Cl. G01N 33/574

(52) U.S. Cl. CPC G01N 33/57488; G01N 2333/968

(57) ABSTRACT

The invention relates to the field of medical diagnostics, immunology and oncology, in particular to the new tumor markers, useful in the early diagnosis of cancer and methods for cancer diagnostics. A new antigen for ELISA to detect antibodies associated with malignancies, namely, mature human plasminogen or human plasminogen fragments containing kringles. We propose a method of diagnosing cancer at an early stage by detecting autoantibodies of type IgA, IgG, IgM to human plasminogen or its fragments in a samples of human blood plasma or sera.

### CROSS-REFERENCE TO RELATED APPLICATIONS

This application is a national stage application of PCT/RU2012/001148 filed on Dec. 29, 2012 which claims priority to Russian application RU2012148244 filed on Nov. 14, 2012 currently issued as a patent RU2522231.

### FIELD OF THE INVENTION

The present invention relates to the field of medical diagnostics, immunology and oncology, in particular to the discovering of new tumor markers. The invention is to the early diagnostics of cancer. More specifically, the present invention relates to one of the new universal tumor markers of neoplastic processes, namely, the autoantibodies against plasminogen or its fragments produced in the human body during the growth of tumor and its progression. The invention also relates to methods for diagnosing cancer by detecting these autoantibodies in a sample of human blood plasma. A Kit of enzyme immunoassay is proposed for detection of autoantibodies. The invention also relates to antigens, which are interacted with human autoantibodies that these antigens will use in the diagnostic of cancer.

### Terminology

Technical and scientific terms used in the description, have the same meaning and value, which are commonly used in the relevant areas of science and technology.

The term “antigen” as used herein, refers to proteins or fragments thereof, capable of binding with antibodies.

The term “kringle” refers to a protein domain that has a structure stabilized by three disulfide bonds.

The term “domain” refers to a section of the protein, which is characterized by certain structural and functional properties.

The term “immunoassay” refers to methods of identifying high-molecular compounds, comprising the steps of: (a) the step of contacting the antigen with a biological sample under conditions suitable for the formation of antigen-antibody complexes, and (b) the stage of detection of these complexes.

The term “tumor marker” refers to a high-molecular compounds defined structure, revealing that in the samples of human tissue is associated with cancer.

The term “epitope” in the present invention refers to a section of the protein molecule that can form a bond with the antibody.

The term “human antibody” refers to an antibody having an amino acid sequence that corresponds to the amino acid sequence of the antibody produced by man.

The term “autoantibodies” (auto-aggressive antibodies, autologous antibodies)—antibodies capable to react with autoantigens, that is, with the body's own antigens.

### BRIEF DESCRIPTION OF DRAWINGS

FIG. 1. The primary structure of Human plasminogen.

FIG. 2. Scheme of transformation of Glu-plasminogen to the heavy (K1-5+30 r) and light chain (micro-plasmin).

### DESCRIPTION OF THE RELATED ART

The search of new tumor markers for the diagnosis of cancer in the early stages of the pathological process is one of important approach in the battle with cancer. The beginning of neoplastic processes may be due to various causes, so specific diagnosis requires to use many different markers with specificity to each type of tumor. Thus, the most useful in the early detection of cancer are the universal tumor markers, the detection of which is associated with a starting neoplastic process any kind.

According to the studies of Folkman, the balance of angiogenic and antiangiogenic systems should be stable for normal development of the organism. During normal processes such as organogenesis in the embryo and wound healing in the adult, angiogenesis provides the necessary vascular support for the newly developing tissue. It is known that tumor tissue contains much more blood vessels and capillaries than the surrounding healthy tissue. For these vessels to the fast-growing tumor cells enter the nutrients and oxygen that need to divide. The tumor growth and lethality are dependent upon angiogenesis and that angiogenesis inhibition suppresses tumor development (Folkman J. Angiogenesis in cancer, vascular, rheumatoid and other disease. Nat Med 1995; 1:27-31, Folkman J. Tumor angiogenesis. Adv Cancer Res 1985; 43:175-203).

It is known a minimal amount of cancer cells are appeared in the tissues, but organism is resistant of them, though some conditions would be suitable for their development (sufficient number of vessels, appropriate microenvironment, and their volume is greater than 2 mm 2) the tumor will begin to grow rapidly.

The role of serine proteases in oncogenesis and other systemic diseases is actively investigated. Secretion of serine proteases by tumor cells leads to the destruction of normal tissue architecture, invasion of these cells into the host tissue. These enzymes, both endogenous and exogenous, can remove from the cell surface specific proteins, which leads to the change in the adhesive properties of cells. Serine proteases secreted from tumor tissue interstitial fluid, can destroy the primary capillary membrane, facilitating the penetration of tumor cells through the vessel wall to the tissues of the host and the formation of metastases. Plasminogen/plasmin takes special attention in the class of serine proteases involved in process of oncogenesis whereas it has antiangiogenic properties too. Angiostatin and some other products of degradation of plasminogen are components of antiangiogenic plasminogen system.

Plasminogen is inactive precursor of plasmin. Plasmin is a endopeptidase with trypsin-like serine protease activity. Physiological action of plasmin is to control the balance of coagulation system. Plasmin is usually performed from the plasminogen by activation of streptokinase, urokinase in vitro. Native glu-plasminogen is readily converted to lys-plasminogen by plasmin hydrolysis of the Lys-77-Lys-78 bond. The system of plasminogen/plasmin is active not only in the process of fibrinolysis, but is closely associated with process of carcinogenesis. It was demonstrated a strong relationship between plasmin and metalloproteases, which are active components in carcinogenesis (Yves A. DeClerck and Walter E. Laug: Plasminogen: Structure, Activation, and Regulation, edited by David M. Waisman. Kluwer Academic/Plenum Publishers, New York, 2003). Apart from a native molecule of plasminogen (plasmin), a whole range of products of its degradation are functionally significant. Effect of degradated forms of plasmin to small molecule substrates may exceed of whole molecule (J G Klys, N V Zaitseva, A I Kizim, SV rope Proteolytic derivatives of plasminogen in the development of cancer, oncology, T 12, No 1, 2010).

In the process of degradation of the molecule plasminogen (plasmin), light and heavy chains can be obtained. The light chain contains the active site of plasmin, which is characteristic for the whole class of serine proteases. The heavy chain contains five kringles (triple disulfide-linked loops) regions. Each of these kringles or combination has its functional specialization. There are variants of the existence of the kringles in plasma: K1-3; K2-3; K1-4; K1-4,5; K1-5 and some single Kringles (Perri S, Martineau D, Francois M, et al. Plasminogen kringle 5 blocks tumor progression by antiangiogenic and proinflammatory pathways, Mol Cancer Ther 2007; 6:441-9). Well known that all Kringles, and combinations thereof are actively involved in angiogenesis and tumorigenesis. (Cao et al., J. Biol. Chem. 271:29461-29467, 1996; U.S. Pat. No. 6,024,688 to Folkman et al.) The functional activity of the first four kringle (K1-4) was widely investigated. The sequence of the kringles 1-4 is named angiostatin. (Francis J. Castellino, Victoria A. Ploplis, Structure and function of the plasminogen/plasmin system, Thromb Haemost 2005; 93:647-54; C. Boccaccio and Paolo M. Comoglio Cancer Res 2005; 65(19):8579-82; Rijken D C, Lijnen H R. New insights into the molecular mechanisms of the fibrinolytic system. J Thromb Haemost 2009; 7:4-13).

### DETAILED DESCRIPTION OF THE INVENTION

Authors performed a preliminary study comparing blood samples of cancer patients and controls using two-dimensional electrophoresis followed by mass spectrometry procedure of interesting spots. Preliminarily, samples were prepared using lys-sepharose. It was found a concentrations of some proteins in cancer patients are significantly much higher than in the controls. 90% of these proteins after mass-spectrometric identification were identified as fragments of plasminogen with a molecular mass of less than 55 kDa and immunoglobulins.

The inventors have suggested that in in the area of tumor growth certain peptides are formed in high concentrations that may lead to the production of autoantibodies. These autoantibodies, in turn, can have an inhibitory effect on angiostatin and other derivatives of plasminogen and thus convert the balance in favor of angiogenic system. The neovessel organization is one of the conditions leading to the rapid growth of the tumor. Experiments performed by the inventors have shown the role of human autoantibodies in early detection of of the tumor growth. There are autoantibodies against their own plasminogen and various products of its degradation, particularly angiostatin. Increased titer of autoantibodies to plasminogen and or its degradation products in the plasma is a marker of tumor at an early stage and measurement of the level of autoantibodies to plasminogen and/or its degradation products in the plasma is diagnostic factor of developing cancer.

Tumors of internal organs do not have clear symptoms at early pathological growth usually. Malignant growth used to begin with chronic inflammation, without striking symptoms. Symptoms depend on the location and size of the cancer, as well as how amazed surrounding organs and tissues of the human body. Already formed a malignant tumor in stage I and II growth is painless and has no pronounced symptoms.

Nonspecific symptoms were taken in attention thorough examination of the patient, including through laboratory studies of blood plasma for early detection of cancer markers of the present invention.

Since autoantibodies are polyclonal and plasminogen molecule contains many epitopes, the inventors propose to use different parts of the plasminogen molecule to determine the titer of autoantibodies for early detection of cancer. Currently, there are no published sources data revealing the correlation between the appearance of autoantibodies in the blood plasma to plasminogen and/or its degradation products in cancer.

Plasminogen is a single-chain glycoprotein present in plasma at a concentration of about 2 mcM (Wahl et al., Thromb. Res. 27:523-535, 1982; Kang et al., Trends Cardiovasc. Med. 90:92-102, 1999). Plasminogen contains 791 amino acid residues and 24 disulfide bonds. Protein consists of a single polypeptide chain, where N-terminal amino acid is glutamine, C-terminal asparagine. The structure of the molecule has 2%-3% of carbohydrates, which are localized in the heavy chain. Oligosaccharides attached to Asp288 and Tre345. Plasminogen is a precursor of plasmin which is formed by cleavage of plasminogen between Arg-561 and Val-562 by tissue plasminogen activator or urokinase-type plasminogen activator. In the process of activation of plasminogen bond Arg560-Val561 is cleaved and two chains are formed, light and heavy, connected by disulfide bonds. The light chain (Val561-Asn790) has an active protease center, including the amino acid sequence of serine, histidine, asparagine. The heavy chain of plasmin (Lys78-Arg560) has five triple disulfide-linked loops known as kringle regions—or kringle domains, which is a compact globular structure with a hydrophobic core. These structures are involved in the process of protein interactions in blood clotting. Both plasminogen and plasmin bind to fibrin through amino-terminal kringle regions each of which is a triple loop region formed as a result of disulfide bonds. Kringles of heavy chain named K1, K2, K3, K4, K5. Kringles 1-4 have domains, specific areas, which have a strong affinity for lysine, ε-aminocaproic acid, parabens, and other ω-carbon amino acids having antifibrinolytic properties. Lysine binding sites (LBS) play an important role in the interaction between plasmin (plasminogen) to fibrin and plasmin to inhibitor—a2-AP (antiplasmin). Any fragment of human plasminogen containing any kringles can be used to detect autoantibodies associated with cancer, there is no matter whether a product of cleavage is from natural plasminogen or produced by splitting the plasminogen in vitro (eg, by enzymatic action).

Full-size human plasminogen and various products of its cleavage: light or heavy chain, and any of the fragments containing kringles, can be used as an antigens to produce a set of immunoassay for determination of autoantibodies classes IgG, IgA, and IgM in the samples of human plasma or sera. These antigens are derived from the native Glu-plasminogen, or can be obtained by using gene engineering techniques by recombinant peptide synthesis in eukaryotic and bacterial expression systems. Recombinant antigens are corresponding to amino acid sequence of human plasminogen. In particular, the antigen for the invention disclosed in the method of diagnosis, except for full-plasminogen. It may be the following fragments: Lys-plasminogen, the heavy chain (Glu-H), heavy chain (Lys-H), light chain (L), K1-4 (Tyr80-Ala440), K1-3 (Tyr80-Val338), K1-3 (Tyr80-Val354), K1-4 (Asn60-Pro447), K1-4 (Lys78-Pro447), K1-4 (Lys78- Pro446), K1-4 (Lys78-Lys468), K1-4, 5 (Lys78-Arg530), C4-5 (Val355-Phe546), K1 (Tur80-Glu164), K2-3 (Cys165-Val338), K4 (Val354-Ala440), K5 (Ser441-Fhe546), K5 (Val442-Arg561), miniplasminogen, and any combination thereof, (Table 1).

The inventors discovered and experimentally first confirmed that human plasminogen or its fragments can be used as antigens to determine the titer of autoantibodies in ELISA of human plasma and the result of this reaction can be used to diagnose the presence of cancer. So, as disclosed in the present invention, method of diagnosis is based on a well-defined polypeptides with unique amino acid sequences that any other proteins with identical primary structures and amino acid sequences are identical to those disclosed in the invention. It is clear that an antigens of the present invention can be any polypeptide having partial homology (90% and above) with the claimed polypeptides, since the replacement of individual amino acids does not alter the 3-dimentional structure of Kringle and not an obstacle to the interaction of the antigen-antibody.

Table 1 describes the various polypeptides—derived from human plasminogen. Kringle fragments form that can be used for preparation an enzyme immunoassay with samples of human blood plasma to detect autoantibodies associated with the growth of tumor.

| peptide chain | Mass, kDa | Name | |
|---|---|---|---|
| Glu¹-Asn⁷⁹¹ | 98 | Glu- | SEQ ID NO: 1 |
| | | plasminogen | |
| Lys⁷⁸-Asn⁷⁹¹ | 83, 5-84 | Lys-plasminogen | SEQ ID NO: 2 |
| Glu¹-Arg⁵⁶¹ | 65 | heavy chain (Glu- | SEQ ID NO: 3 |
| | | H) | |
| Lys⁷⁸-Arg⁵⁶¹ | 59 | heavy chain (Lys- | SEQ ID NO: 4 |
| | | H) | |
| Val⁵⁶²-Asn⁷⁹¹ | 25 (26, 3) | light chain (L) | SEQ ID NO: 5 |
| Tyr⁸⁰-Ala⁴⁴⁰ | 51-54 | K1-4(Tyr⁸⁰- | SEQ ID NO: 6 |
| | | Ala⁴⁴⁰) | |
| Tyr⁸⁰-Val³³⁸ | 41 | K1-3(Tyr⁸⁰- | SEQ ID NO: 7 |
| | | Val³³⁸) | |
| Tyr⁸⁰-Val³⁵⁴ | 44 | K1-3(Tyr⁸⁰- | SEQ ID NO: 8 |
| | | Val³⁵⁴) | |
| Asn⁶⁰-Pro⁴⁴⁷ | 55 | K1-4(Asn⁶⁰- | SEQ ID NO: 9 |
| | | Pro⁴⁴⁷) | |
| Lys⁷⁸-Pro⁴⁴⁷ | 58 | K1-4(Lys⁷⁸- | SEQ ID NO: 10 |
| | | Pro⁴⁴⁷) | |
| Lys⁷⁸-Pro⁴⁴⁶ | 58 | K1-4 (Lys⁷⁸- | SEQ ID NO: 11 |
| | | Pro⁴⁴⁶) | |
| Lys⁷⁸-Lys⁴⁶⁸ | 61 | K1-4(Lys⁷⁸- | SEQ ID NO: 12 |
| | | Lys⁴⁶⁸) | |
| Lys⁷⁸-Arg⁵³⁰ | 66, 60, 57 | K1-4,5 (Lys⁷⁸- | SEQ ID NO: 13 |
| | | Arg⁵³⁰) | |
| Val³⁵⁵-Phe⁵⁴⁶ | | K4-5(Val³⁵⁵- | SEQ ID NO: 14 |
| | | Phe⁵⁴⁶) | |
| Tyr⁸⁰-Glu¹⁶⁴ | 9, 81 | K1(Tyr⁸⁰-Glu¹⁶⁴) | SEQ ID NO: 15 |
| Glu¹-Asn⁷⁹¹ | 98 | Glu- | SEQ ID NO: 1 |
| | | plasminogen | |
| Cys¹⁶⁵-Val³³⁸ | | K2-3(Cys¹⁶⁵- | SEQ ID NO: 16 |
| | | Val³³⁸) | |
| Val³⁵⁴-Ala⁴⁴⁰ | 10-12 | K4(Val³⁵⁴-Ala⁴⁴⁰) | SEQ ID NO: 17 |
| Ser⁴⁴¹-Fhe⁵⁴⁶ | 12 | K5(Ser⁴⁴¹-Fhe⁵⁴⁶) | SEQ ID NO: 18 |
| Val⁴⁴²-Arg⁵⁶¹ | | K5(Val⁴⁴²-Arg⁵⁶¹) | SEQ ID NO: 19 |
| Val⁴⁴²-Asn⁷⁹¹ | (40) 38 | miniplasminogen | SEQ ID NO: 20 |

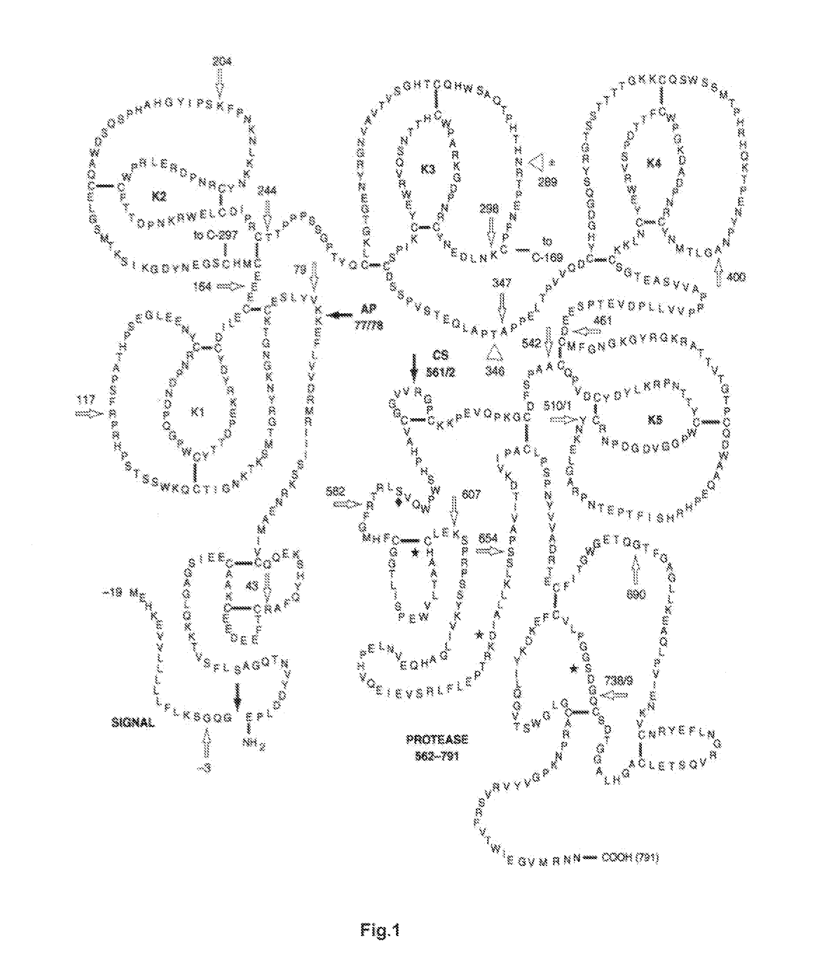

FIG. 1 shows the primary structure of Human plasminogen.

Filled arrows identify the cleavage sites for: (a) the release of the signal peptide between residues—1 and 1, which is required for the generation of the mature form of the protein; (b) the release of the activation peptide (Glu′-Lys⁷⁷) resulting in the conversion of Glu′-Pg to Lys⁷⁸-Pg or Glu′-Pm to Lys⁷⁸-Pm; (c) the activation of Human plasminogen to plasmin (CS) at the Arg⁵⁶¹-Val⁵⁶² peptide bond. Unfilled arrows identify introns in the gene sequence. Triangles locate the N-linked oligosaccharide site at sequence position 289 and the O-linked glycan at position 346. The catalytic triad, His⁶⁰³, Asp⁶⁴⁶, and Ser⁷⁴¹, is also indicated (*). Disulfide bonds are depicted by heavy bars. ♦ phosphorylation site alanine—ala—A; arginine—arg—R; asparagine—asn—N; aspartic acid—asp—D; cysteine—cys—C; glutamine—gln—Q; glutamic acid—glu—E; glycine—gly—G; histidine—his—H; isoleucine—ile—I; leucine—leu—L; lysine—lys—K; methionine—met—M; phenylalanine—phe—F; proline—pro—P; serine—ser—S; threonine—thr—T; tryptophan—trp—W; tyrosine—tyr—Y; valine—val—V.

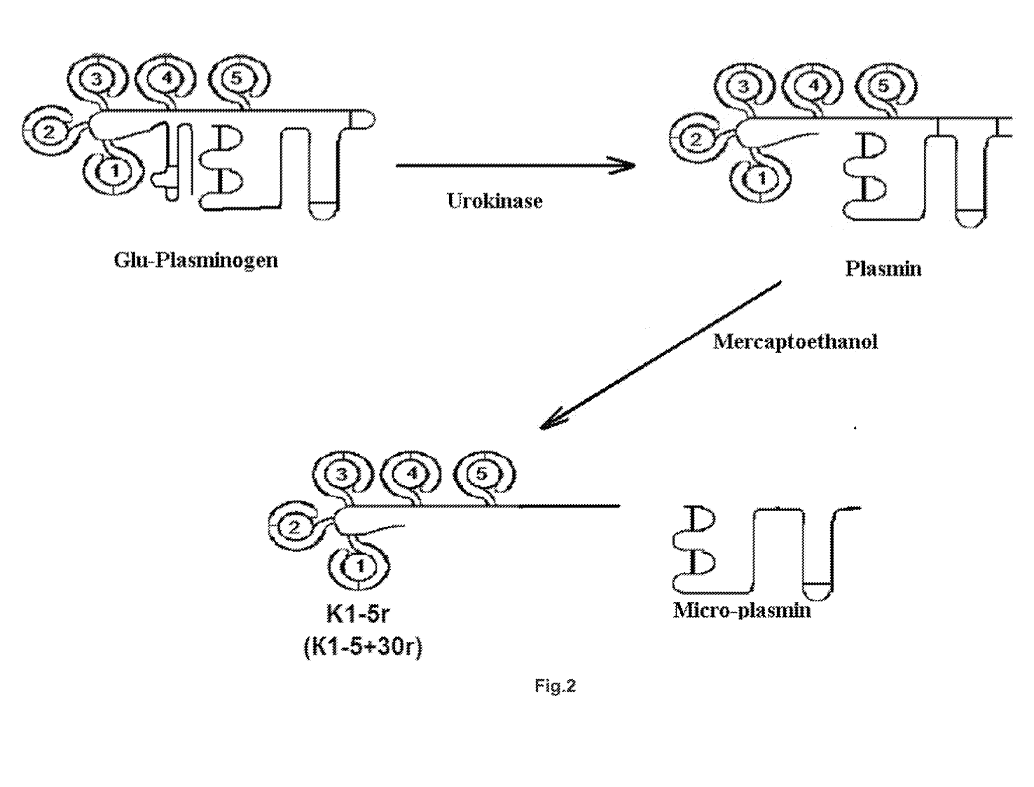

FIG. 2 shows the scheme of transformation of Glu-plasminogen to the heavy (K1-5+30 r) and light chain (micro-plasmin).

### BRIEF SUMMARY OF THE INVENTION

Human plasminogen and its fragments disclosed herein (Table 1) were purified from blood plasma and used as antigens to create enzyme immunoassay for the determination of levels of autoantibodies class IgG, IgA, IgM to plasminogen/or its fragments in the blood of patients with various cancers, confirmed by alternative methods of diagnostics. According to the fact that the growth of all solid tumors is accompanied by increased growth of vessels and capillars in the area of the tumor the concentration of plasminogen and its products of degradation are used to increase in this area, that leads to the synthesis of autoantibodies to these products. Thus, the undoubted advantage of the present invention disclose the common marker of early diagnostic of cancer which is to the appearance of autoantibodies to plasminogen and its products of degradation in humans associated with the development of a tumor any kind.

Isolation of Antigens for ELISA.

The method for preparing of heavy chain (Glu-H) Glu1-Arg561 and light chain (L) Val562-Asn791 of human plasminogen.

The base of the method consists in the activation of plasminogen to plasmin, followed by reduction of S-S bonds between heavy and light chains in conditions that exclude autolysis, and using affinity chromatography on Lys-Sepharose 4B for the following separation. Urokinase cleave Arg561-Val562 bond in plasminogen. The resulting plasmin cut 77-78 bond and cleaved N-terminal peptide (1-77) is out. Mercaptoethanol reduct two bonds between Cys558-Cys566 and Cys548-Cys666, linking the heavy and light chains.

### First Step:

Glu-Plasminogen was isolated from frozen donor human plasma by affinity chromatography on Lys-Sepharose 4B at 4° C., pH 8.0. Blood plasma was thawed in the presence of aprotinin, centrifuged 30 min at 4° C. and diluted 2-fold to 0.02 M phosphate buffer, pH 8.0, containing 20 KIU/ml aprotinin. Prepared plasma applied into a column with Lys-Sepharose 4B, equilibrated with 0.1 M K-phosphate buffer, pH 8.0, containing 20 KIU/ml aprotinin. The column was washed from unbound protein to 0.3 M phosphate buffer, pH 8.0, containing 20 KIU/ml aprotinin, overnight to absorbance at A280=0.05-0.01. Glu-Plasminogen was eluted with a solution of 0.2 M 6-aminocaproic acid, 0.1 M K-phosphate buffer, pH 8.0, containing 20 KIU/ml aprotinin. Fractions containing protein were pooled and subjected to further purification by precipitation (NH4) 2SO4 (0.31 g/ml protein solution). The precipitate was allowed to stand at 4° C. for 18-24 hours and then separated by centrifugation and dissolved in 0.05 M Tris-HCl buffer, pH 8.0 to a concentration of about 1.5-2.0 mg/ml. Purified Glu-Plasminogen dialyzed at 4° C. against water (pH 8.0) and lyophilized.

### Second Step:

To a solution of Glu-plasminogen (5 mg/ml) in 0.05 M Tris-HCl buffer, pH 8 8, containing 0.02 M L-lysine, 0.15 M NaCl, 20% glycerol, and 6000 KIU/ml aprotinin urokinase was added to a final concentration of 600 IU/ml and incubated for 4 h at 37° C. The complete of conversion of Glu-plasminogen to plasmin was monitored by an increase to a maximum rate of hydrolysis of plasmin specific substrate S-2251 (HD-Val-Leu-Lys p-nitroanilide, “Sigma”, USA) in samples taken from the reaction mixture.

Third stage

The reduction of S-S-bonds between heavy and light chains of plasmin.

Mercaptoethanol was added to the solution of plasmin to a final concentration of 0.25 mM and incubated under nitrogen in the dark for 20 minutes at room temperature. In the result, free SH-groups were blocked by adding freshly prepared solution iodoacetic acid in 0.1 M Na-phosphate buffer, pH 8.0 (final concentration of 0.315 M) and incubated for 20 min.

Fourth Stage:

The separation of heavy and light chains of plasmin by column chromatography on Lys-Sepharose 4B.

The reaction mixture was diluted to a concentration of 1 mg/ml of protein by 0.1 M Na-phosphate buffer, pH 8.0, containing 20 KIU/ml aprotinin and applied to Lys-Sepharose 4B column equilibrated with the same buffer. Chromatography was performed at 25° C. The heavy chain of plasmin has kringles 1-5 and 30 amino acid residues of the connecting peptide. It was adsorbed on the sorbent, but light chain is eluted with equilibration buffer. Heavy chain (MR˜56-57 kDa) was eluted by 0.2 M solution of 6-aminocaproic acid in 0.1 M Na-phosphate buffer, pH 8.0. The pooled fractions were dialyzed against water (pH˜8.0) and lyophilized.

The purity and molecular weight of the protein was investigated by 12% SDS-polyacrylamide gel electrophoresis.

Furthermore, the absence of amidase activity (for S-2251) before and after incubation with streptokinase its solution indicated that the heavy chain does not contain trace concentrations of miniplasminogen, which may not show up during electrophoresis.

The purification of Lys-plasminogen (Lys78-Asn791) and heavy chain (Lys-H Lys78-Arg561) was performed by the same method, but without inhibitor -aprotinin.

Isolation of miniplasminogen (Val442-Asn791). Miniplasminogen consist of K5 and light chain. Its sequence starts from Val442 to Asn791. Miniplasminogen obtained by incubation of Lys-plasminogen (Lys78-Asn791) with elastase followed by gel filtration on G-75 Sephadex.

Isolation of Kringle K1-4, 5 (Lys78-Arg530) was performed according to the method described in Cao R., Wu H. L., Veitonmaki N., Linden P., Farnedo J., Shi C.Y., and Cao Y, (1999) Proc. Natl. Acad. Sci. USA, 96, 5728-5733, with some modifications.

Glu-plasminogen (10 mg/ml) was activated with urokinase (600 ME/ml) in 0.05 M phosphate buffer, pH 9.0, containing 0.02 M L-lysine and 0.1 M NaCl, at 37° C. Complete conversion of plasminogen to plasmin was monitored by the increase in the amidase activity of the solution to the maximum value. An equal volume of 0.2 M glycerol buffer, pH 12.0 was added to a solution of plasmin and incubated for 18 h at 25 C, pH of 10.5. The reaction mixture was diluted 5-fold with buffer containing 0.1 M phosphate buffer, pH 8.0, and 40 KIU/ml aprotinin, and applied to a column of Lys-Sepharose 4B equilibrated with the same buffer. After the following out of microplasminogen, adsorbed K1-4,5 was eluted from the column with 0.2 M solution of 6-aminocaproic acid in 0.1 M phosphate buffer, pH 8.0 and 40 KIU/ml aprotinin, dialyzed against water and lyophilized. The purity of the substance was checked by 12% SDS-polyacrylamide gel electrophoresis.

Isolation of kringle K1-4 (Tyr80-Ala440) and K1-3 (Tyr80-Val338) K4-5 (Val355-Phe546) was performed using elastase treatment of Glu-plasminogen by the method described in the works of Cao Y., Ji R. W., Davidson D., Schaller J., Marti D., Sohndel S., McCanse S. G., O'Reilly M. S., Llinas M., and Folkman J. (1996) J. Biol. Chem., 271, 29461-29467. Glu-plasminogen was incubated with elastase at a ratio of 50:1 (M/M) in a buffer containing 0.05 M Tris-HCl, pH 8.5, 0.5 M NaCl, and 200 KIU aprotinin, for 5 hours at room temperature. The reaction was stopped by adding PMFS to maintain its concentration 1 mM for 40-50 min. Then gel-filtration on a column of Sephadex G-75 was performed to separate low and high molecular proteins. Protein fractions of the second peak containing K1-3, K1-4, K4-5 and miniplasminogen was applied to an affinity column with Lys-Sepharose 4B equilibrated with buffer containing 0.05 M Tris-HCl, pH 8.5, 0.15 M NaCl. After the flowing throw miniplasminogen which is not adsorbed on the Lys-Sepharose 4B, adsorbed fragments K1-3, K1-4 and K4-5 was eluted with a solution of 0.2 M 6-aminocaproic acid in the same buffer, dialyzed against a buffer containing 0.02 M Tris-HCl, pH 8.0, and applied to a column of heparin-agarose equilibrated with the same buffer. After elution of unbound fragment K1-4 and K4-5 with the buffer, the fragment K1-3 was eluted with a solution of 0.25 M KCl in the same buffer. The purified fragment K1 -3 was dialyzed against water and lyophilized. K1-4 and K4-5 were separated by gel filtration on Sephadex G-75.

Kringles K5 (Ser449 (Pro452)-Fhe546), K1-3 (Tyr80-Val338), K-4 (Val335-Ala440) were prepared according to the work of Cao, Y., Chen, A., An, S. S. A., Ji, R. W., Davidson, D., and Llinas, M. (1997) J. Biol. Chem. 272, 22924-22928). The method is to digest by elastase Lys-plasminogen (Lys78-Asn791). After processing elastase mixture was applied to a column of Mono-S (Bio-Rad) equilibrated with buffer containing 20 mM NaOAc, pH 5.0. Fragments of plasminogen were eluted by gradient 1 M KCl in buffer containing 20 mM NaOAc, pH 5.0. We used a gradient of 0-20%, 20-50%, 50-70% and 70-100%. K-5 release to 50%. For this scheme, but in a another gradient K-4 (Val335-Ala440) and kringle K1-3 (Tyr80-Val354) were obtained.

The method of isolation of K5 (Val442-Arg561) is to digesting by elastase of miniplazminogen (Val442-Asn791) containing 5-kringle of heavy chain following by digesting of the fragment by pepsin and then using the gel filtration and ion exchange chromatography according to the work (Theresa Thewes, Vasudevan Ramesh, Elcna L. Simplaceanu and Miguel Llinfis, Isolation, purification and I H-NMR characterization of a kringle 5 domain fragment from human plasminogen (Biochimica et Biophysica Acta 912 (1987), 254-269).

Kringle K1-4 (Lys78-Pro446) and K1-4 (Lys78-Lys468) was prepared according to the method with metalloproteinases (Patterson, B. C. and Sang, Q. A. (1997) J. Biol. Chem. 272, 28823-28825).

Kringle K1-4 (Asn60-Pro447) obtained by the method with metalloproteinases (Lijnen, H. R., Ugwu, F., Bini, A., and Collen, D. (1998) Biochemistry 37, 4699-4702).

Kringle K1 (Tur80-Glu164) and K2-3 (Cys165-Val338) were isolated from the K1-3 (Tyr80-Val338) by treating of pepsin (or protease s.aureus V8) with a further separation on lys-Sepharose and gel filtration on Sephadex G-75.

### Preparation of Diagnostic Test Kits for ELISA of Autoantibodies

Glu-plasminogen or its fragments having at least one kringle were used as Antigens for ELISA of autoantibodies. Different types of antigens used in ELISA are listed in Table 1. Their primary amino acid sequence shown in the sequence listing.

The antigen was diluted in 0.1 M carbonate-bicarbonate buffer pH 9.6 in the maximum concentration of 5 μg/ml for molecules with a molecular weight of more than 25 kDa and 10 μg/ml for molecules with a molecular weight less than 25 kDa. These dilutions of antigen were used to identify all types of immunoglobulins.

PBS (phosphate buffered saline, phosphate salt solution):

0.14 M NaCl; 0.003 M KCl, 0.005 M Na2HPO4, 0.002 M KH2PO4

Preparation 1 L 10× PBS:

80 g-NaCl 2 g-KCl 18 g-Na2HPO4 2 g-KH2PO4

Substrate buffer solution (pH 4.3): 31 mM citric acid, 0.05 N NaOH, 3 mM H2O2

TMB solution: 5 mM 3,3′, 5,5′-tetramethylbenzidine in 70% DMSO

Chrornogenic substrate solution (prepared before use): Mixed 4 parts of the substrate buffer solution and one part of the TMB solution.

To prepare a kit for ELISA was performed pre-immobilized antigen. To immobilize the antigen can be used different types of material such as nitrocellulose, glass beads or other particles that can absorb proteins, immunological plastic strips or plates. We used for experiments immunological plastic strips (Nunc). To each well was diluted into 100 μL antigen solution. Incubation was carried out for 14-16 hours at 37° C. in a humidified chamber. The antigen solution was removed by shaking out, and then wells washed twice by a solution containing a PBS with 0.05% Tweeen-20, 200 μL/well to remove unbound antigen. For the block was used solution of 1% gelatin in PBS, 200 μL/well, with incubation for 1.5-2 hours at room temperature. After incubation, the blocking fluid was removed, the plate was dried overnight at room temperature on air.

Control probes and tested samples were diluted in 50-fold by diluent (0.5% gelatin, 0.001 M EDTA in PBS), then were poured 100 μL into appropriate wells and incubated for 1 h at 37° C. After incubation, the solution were removed, the plate was washed four times with washing buffer (PBS with 0.05% Tween-20), each time carefully removing the contents of the wells. Working dilution of conjugate (to determine IgG, IgA, IgM as conjugate used respectively Mab Fc IgG-peroxidase, Mab Fc IgA-peroxidase, Mab IgM-peroxidase) were added into the appropriate wells of 100 mcl/well and then incubated for 1 h at 37° C. Unbound components were removed 4 times by washing of wash solution. Then in all the wells were added 100 μL of substrate-chromogen solution, and incubated for 15 minutes at 370 C. The reaction was stopped by adding to wells 100 μL of stop solution (2M H2SO4). Photometry was performed on vertical scanning photometer “UNIPLAN” (company “Picon”, Russia) with a wavelength of 450 nm.

The Diagnostic of Cancer by Enzyme Immunoassay Detection of Autoantibodies to Human Plasminogen and/or its Fragments

Blood samples were taken from patients cubital vein using vacutaners with EDTA. The samples were then centrifuged at 3000 r/min for 15 min. Plasma was poured into tubes in 100 μL., Frozen and stored at −40 C.

The control group has plasma samples taken from 30 healthy men and 30 healthy women. Each sample was negative in tests for hepatitis A, B and C virus, HIV, tuberculosis and syphilis.

The level of autoantibodies IgG and IgA in the control samples was measured using the ELISA kit, according to the described method. Dilution of control plasma samples was chosen so that the optical density was less than 0.2.

The dilution of samples in ELISA was established of 1/50 for each tested samples, which was subsequently used for the analysis of all samples. The antigen was used as a whole molecule of glu-plasminogen, as its fragments. For accuracy, the determination of each sample was tested in duplicate. After measuring 30 male and 30 female control samples was calculated the average optical density for each of the control group used for testing with various fragments of plasminogen or full plasminogen molecule as a ligand.

ELISA test samples was performed with each individual antigen. Number of samples above the average in the male control group was within 2% to 5%, while in the women's within 3% to 6% when tested with all antigens investigated. For a comparative study of the control group to samples of cancer and other diseases for the control group were taken 5 samples with indicators of optical density, not more than 5% of the average. These five samples were mixed into one pooled sample—control sample (K), used as a reference level of autoantibodies to plasminogen or its fragments. The samples of control were different to the study of antibodies IgG, IgA and IgM.

To assess the effectiveness of various fragments of plasminogen for the early diagnostic of cancer was used the samples of plasma of patients with various forms of cancer (Table 2).

TABLE 2
Prostate Cancer

| No | Age | Diagnosis | Biopsy | |
|---|---|---|---|---|
| 1 | 68 | T2cNoMo | small acinar adenocarcinoma amid mixed | PSA - 35 ng/ml |
| | | | hyperplasia and chronic prostatitis. Gleason | |
| | | | score 2 (1 + 1) | |
| 2 | 58 | T2cNxMo | acinar adenocarcinoma. Gleason score 6 (4 + | PSA -9 ng/ml |
| | | | 2) | |
| 3 | 69 | T3NxMo | adenocarcinoma | |
| 4 | 67 | T2aNoMo | Focuses of small acinar adenocarcinoma, | PSA 7 ng/ml |
| | | | Gleason score 4 points (2 + 2), mixed | |
| | | | hyperplasia, chronic prostatitis | |
| 5 | 64 | T2NxMo | small acinar adenocarcinoma, Gleason | PSA - 26 ng/ml |
| | | | score 5 (3 + 2) | |
| 6 | 63 | T2cNoMo | small acinar adenocarcinoma, Gleason | [symbol] CA-12 ng/ml |
| | | | score 7 (3 + 4) | |
| 7 | 62 | ToNoMo | small acinar adenocarcinoma, Gleason | PSA - 35 ng/ml |
| | | | score 3 (1 + 2) | |
| 8 | 78 | T2NxMo | small acinar adenocarcinoma, Gleason | PSA - 25 ng/ml |
| | | | score 5 (3 + 2) | |
| 9 | 72 | T2NxMo | small acinar adenocarcinoma, Gleason | PSA - 50 ng/ml |
| | | | score 5 (3 + 2) | |
| 10 | 62 | T2cNoMo | small acinar adenocarcinorna, Gleason | [symbol] CA-10 ng/ml |
| | | | score 5 (2 + 3) | |
| 11 | 78 | T3NxMx | adenocarcinoma | PSA - 20 ng/ml |
| 12 | 73 | T2NxMo | small acinar adenocarcinoma, Gleason | PSA - 55 ng/ml |
| | | | score 7 (4 + 3) | |
| 13 | 75 | T2aN0Mo | small acinar adenocarcinoma, Gleason | PSA - 11 ng/ml |
| | | | score 4 points (2 + 2) | |
| 14 | 73 | T3aNxMx | small acinar adenocarcinoma, with | PSA - 44 ng/ml |
| | | | hyperplasia and chronic prostatitis | |
| 15 | 60 | T2N0M0 | small acinar adenocarcinoma, Gleason | PSA - 67 ng/ml |
| | | | score 7 (4 + 3) | |
| 16 | 66 | T2NxMx | Large acinar adenocarcinoma, Gleason | PSA - 57 ng/ml |
| | | | score 5 (3 + 2) | |
| 17 | 71 | T2bN0M1 | acinar adenocarcinoma, Gleason score 7 (3 + | PSA - 70 ng/ml |
| | | | 4) | |
| 18 | 70 | T3N1M0 | small acinar adenocarcinoma, Gleason | PSA - 120 ng/ml |
| | | | score 9 (4 + 5) | |
| 19 | 73 | T3N1M0 | small acinar adenocarcinoma, Gleason | PSA - 30 ng/ml |
| | | | score 3 (3 + 3) | |
| 20 | 78 | T3N1M0 | acinar adenocarcinoma, Gleason score 7 (3 + | PSA - 70 ng/ml |
| | | | 4) | |
| 21 | 81 | T2N0M0 | acinar adenocarcinoma, Gleason score 7 (3 + | PSA - 15 ng/ml |
| | | | 4) | |
| 22 | 78 | T2N0M0 | small acinar adenocarcinoma, Gleason | PSA - 70 ng/ml |
| | | | score 7 (4 + 3) | |
| 23 | 46 | T2N0M0 | small acinar adenocarcinoma, Gleason | PSA - 35 ng/ml |
| | | | score 6 (3 + 3) | |
| 24 | 71 | T2cN0M0 | Adenocarcinoma Gleason score 5 (2 + 3) | PSA - 18 ng/ml |
| 25 | 59 | T3NxM1 | Adenocarcinoma, Gleason score 8 (4 + 4) | PSA - 225 ng/ml |
| 26 | 64 | T2N0M0 | small acinar adenocarcinoma, Gleason | PSA - 16 ng/ml |
| | | | score 5 (3 + 2) | |
| 27 | 73 | T3aNxMx | small acinar adenocarcinoma, with | PSA - 44 ng/ml |
| | | | hyperplasia and chronic prostatitis, Gleason | |
| | | | score 5 (3 + 2) | |
| 28 | 65 | T3NxM1 | small acinar adenocarcinoma, Gleason | PSA - 163 ng/ml |
| | | | score 9 (4 + 5) | |
| 29 | 62 | T2aNoMo | Focuses small acinar adenocarcinoma | PSA - 70 ng/ml |
| 30 | 64 | T2aNxMx | Focuses small acinar adenocarcinoma, | PSA - 14 ng/ml |
| | | | hyperplasia and chronic prostatitis | |

Lung Cancer

| No | Age, sex | Diagnosis | Biopsy |
|---|---|---|---|
| 1 | 77 f | Cancer of the upper lobe of the | moderately differentiated |
| | | left lung, T2N0M0, stage IB | adenocarcinoma |
| 2 | 71 f | Cancer of the upper lobe of the | moderately differentiated |
| | | left lung, T2N0M0, stage IIB | squamous cell carcinoma |
| 3 | 80 M | Cancer of the upper lobe of the | moderately differentiated |
| | | left lung, T2N1M0, stage IIB | squamous cell carcinoma |
| 4 | 65 M | Cancer of the right upper lobe, | moderately differentiated |
| | | T2N0M0, stage IB | squamous cell carcinoma |
| 5 | 63 M | Cancer of the upper lobe of the | moderately differentiated |
| | | left lung, T2N1M0, stage IIB | glandular squamous cell |
| | | | carcinoma |
| 6 | 70 M | Cancer of the lower lobe of the | small-cell lung carcinoma |
| | | right lung, T2N0M0, stage IB | |
| 7 | 71 M | Cancer of the right upper lobe | moderately differentiated |
| | | T2N0M0, stage IB | glandular squamous cell |
| | | | carcinoma |
| 8 | 71 f | Cancer of the upper lobe of the | small-cell lung carcinoma |
| | | left lung, T4N2M0, stage IIIB | |
| 9 | 66 M | Cancer of the lower lobe of the | small-cell lung carcinoma |
| | | right lung, T2N1M1, stage IV, | |
| | | with liver metastases | |
| 10 | 52 M | Cancer of the right upper lobe | small-cell lung carcinoma |
| | | T2N2M0, stage IIIA | |
| 11 | 58 f | Cancer of the lower lobe of the | moderately differentiated |
| | | right lung, T1N0M0, stage IA | squamous cell carcinoma |
| 12 | 57 f | Cancer of the right upper lobe | small-cell lung carcinoma |
| | | T2N0M0, stage IB | |
| 13 | 55 M | Cancer of the lower lobe of the | small-cell lung carcinoma |
| | | right lung, T2N0M0, stage IB | |
| 14 | 68 f | Cancer of the upper lobe of the | moderately differentiated |
| | | left lung, T4N2M0, stage IIIB | squamous cell carcinoma |
| 15 | 65 f | Cancer of the right upper lobe, | Moderately differentiated |
| | | T2N0M0, stage IB | squamous cell carcinoma |
| 16 | 65 M | Cancer of the lower lobe of the | small-cell lung carcinoma |
| | | right lung, T2N0M0, stage IB | |
| 17 | 47 f | Cancer of the lower lobe of the | moderately differentiated |
| | | right lung, T2N0M0, stage IA | adenocarcinoma |
| 18 | 31 M | Cancer of the lower lobe of the | small-cell lung carcinoma |
| | | right lung, T2N1M1, stage | |
| | | Cancer of the upper lobe of the | |
| | | left lung, T4N2M0, stage IVB | |
| | | with metastases | |
| 19 | 54 f | Cancer of the upper lobe of the | small-cell lung carcinoma |
| | | left lung, T4N2M0, stage IIIB | |
| 20 | 54 f | Cancer of the right upper lobe | small-cell lung carcinoma |
| | | T2N2M0, stage IIIA | |

| | | | |
|---|---|---|---|
| No | Age | Diagnosis | Biopsy |

Breast Cancer

| | | | | |
|---|---|---|---|---|
| 1 | 63 | Cancer of the right breast, | Infiltrative ductal carcinoma, | CA 15-3 120 units/mL |
| | | T2N0M0, stage IIB | grade II (6 points) | |
| 2 | 47 | Cancer of the left breast, | Infiltrative ductal carcinoma, | CA 15-3 160 units/mL |
| | | T2N1M0, stage IIA | grade II (6 points)) | |
| 3 | 52 | Cancer of the right breast, | Infiltrative ductal carcinoma, | CA 15-3 140 units/mL |
| | | T2N0M0, stage IIB | grade II (6 points) | |
| 4 | 65 | Cancer of the left breast, | Infiltrative ductal carcinoma, | CA 15-3 110 units/mL |
| | | T4N1M0, stage IIIB | grade II (6 points) | |
| 5 | 59 | Cancer of the left breast, | Infiltrative ductal carcinoma, | CA 15-3 140 units/mL |
| | | T2N1M0, stage IIB | grade II (6 points) | |
| 6 | 72 | Cancer of the left breast, | Infiltrative ductal carcinoma, | CA 15-3 160 units/mL |
| | | T2N2M0, stage IIIA | grade III (8 points) | |
| 7 | 54 | Cancer of the left breast, | Infiltrative ductal carcinoma, | CA 15-3 90 units/mL |
| | | T1N0M0, stage I | grade I (5 points) | |
| 8 | 73 | Cancer of the right breast, | Infiltrative ductal carcinoma, | CA 15-3 100 units/mL |
| | | T2N0M0, stage IIB | grade II (7 points) | |
| 9 | 48 | Cancer of the right breast, | Infiltrative ductal carcinoma, | CA 15-3 170 units/mL |
| | | T4N0M0, stage IIIB | grade II (7 points) | |
| 10 | 42 | Cancer of the left breast, | Infiltrative ductal carcinoma, | CA 15-3 60 units/mL |
| | | T4N1M0, stage IIIB | grade II (7 points) | |
| 11 | 61 | Cancer of the right breast, | Infiltrative ductal carcinoma, | CA 15-3 130 units/mL |
| | | T2N0M0, stage IIB | grade II (6 points) | |
| 12 | 46 | Cancer of the left breast, | Infiltrative ductal carcinoma, | CA 15-3 150 units/mL |
| | | T3N1M0, stage IIIA | grade II (7 points) | |
| 13 | 47 | Cancer of the left breast, | Infiltrative ductal carcinoma, | CA 15-3 100 units/mL |
| | | T2N0M0, stage IIA | grade I (5 points) | |
| 14 | 60 | Cancer of the right breast, | Infiltrative ductal carcinoma, | CA 15-3 180 units/mL |
| | | T2N0M0, [symbol] IIB | grade II (7 points) | |
| 15 | 62 | Cancer of the right breast, | Infiltrative ductal carcinoma, | CA 15-3 90 units/mL |
| | | T2N0M0, stage IIB | grade II (6 points) | |
| 16 | 47 | Cancer of the left breast, | Infiltrative ductal carcinoma, | CA 15-3 140 units/mL |
| | | T4N1M0, stage IIIB | grade II (6 points) | |
| 17 | 28 | Cancer of the left breast, | Infiltrative ductal carcinoma, | CA 15-3 100 units/mL |
| | | T4N1M0, stage IIIB | grade II (6 points) | |
| 18 | 61 | Cancer of the left breast, | Infiltrative ductal carcinoma, | CA 15-3 100 units/mL |
| | | T4N1M0, stage IIIB | grade II (7 points) | |
| 19 | 69 | Cancer of the right breast, | Infiltrative ductal Carcinoma, | CA 15-3 110 units/mL |
| | | T4N1M0, stage IIIB | grade III (8 points) | |
| 20 | 50 | Cancer of the left breast, | Infiltrative ductal carcinoma, | CA 15-3 110 units/mL |
| | | T2N1M0, stage IIB | grade II (6 points) | |
| 21 | 48 | Cancer of the right breast, | Infiltrative ductal carcinoma, | CA 15-3 90 units/mL |
| | | T2N1M0, stage IIB | grade II (7 points) | |
| 22 | 80 | Cancer of the left breast, | Infiltrative ductal carcinoma, | CA 15-3 70 units/mL |
| | | T1N1M0, stage IIA | grade II (6 points) | |
| 23 | 60 | Cancer of the left breast, | Infiltrative ductal carcinoma, | CA 15-3 140 units/mL |
| | | T2N1M0, stage IIB | grade II (7 points) | |
| 24 | 62 | Cancer of the right breast, | Infiltrative ductal carcinoma, | CA 15-3 100 units/mL |
| | | T2N1M0, stage IIB | grade II (6 points) | |
| 25 | 54 | Cancer of the right breast, | Infiltrative ductal carcinoma, | CA 15-3 90 units/mL |
| | | T4N1M0, stage IIIB | grade II (7 points) | |
| 26 | 75 | Cancer of the left breast, | Infiltrative ductal carcinoma, | CA 15-3 120 units/mL |
| | | T2N2M0, stage IIIA | grade III (8 points) | |
| 27 | 67 | Cancer of the right breast, | Infiltrative ductal carcinoma, | CA 15-3 150 units/mL |
| | | T4N1M0, stage IIIB | grade II (7 points) | |
| 28 | 67 | Cancer of the left breast, | Infiltrative ductal carcinoma, | CA 15-3 110 units/mL |
| | | T2N1M0, stage IIB | grade II (6 points) | |
| 29 | 61 | Cancer of the left breast, | Infiltrative ductal carcinoma, | CA 15-3 180 units/mL |
| | | T2N1M0, stage IIB | grade II (6 points) | |
| 30 | 39 | Cancer of the right breast, | Infiltrative ductal carcinoma, | CA 15-3 160 units/mL |
| | | T2N2M0, stage IIIA | grade II (7 points) | |
| 31 | 57 | Cancer of the right breast, | Infiltrative ductal carcilnoma, | CA 15-3 120 units/mL |
| | | T2N1M0, stage IIB | grade III (8 points) | |
| 32 | 70 | Cancer of the left breast, | Infiltrative ductal carcinoma, | CA 15-3 200 units/mL |
| | | T2N1M0, stage IIB | grade II (6 points) | |
| 33 | 69 | Cancer of the left breast, | Infiltrative ductal carcinoma, | CA 15-3 180 units/mL |
| | | T4N2M0, stage IIIB | grade III (8 points) | |
| 34 | 49 | Cancer of the right breast, | Infiltrative ductal carcinoma, | CA 15-3 220 units/mL |
| | | T2N2M0, stage IIIA | grade III (8 points) | |
| 35 | 61 | Cancer of the left breast, | Infiltrative ductal carcinoma, | CA 15-3 140 units/mL |
| | | T2N2M1, stage IV | grade III (8 points) | |
| 36 | 43 | Cancer of the left breast, | Infiltrative ductal carcinoma, | CA 15-3 60 units/mL |
| | | T4N1M0, stage IIIB | grade II (7 points) | |
| 37 | 50 | Cancer of the right breast, | Infiltrative ductal carcinoma, | CA 15-3 200 units/mL |
| | | T2N1M0, stage IIB | grade II (7 points) | |
| 38 | 85 | Cancer of the left breast, | Infiltrative ductal carcinoma, | CA 15-3 250 units/mL |
| | | T2N1M0, stage IIB | grade II (7 points) | |
| 39 | 47 | Cancer of the left breast, | Infiltrative ductal carcinoma, | CA 15-3 150 units/mL |
| | | T4N1M0, stage IIIB | grade II (6 points) | |
| 40 | 55 | Cancer of the left breast, | Infiltrative ductal carcinoma, | CA 15-3 110 units/mL |
| | | T4N2M0, stage IIIB | grade II (6 points) | |

Ovarian cancer

| | | | | |
|---|---|---|---|---|
| 1 | 71 | T3cNoMo, stage IIIC | Serous cystadenocarcinoma | CA125-428 units/mL |
| 2 | 62 | T3N1M0, stage IIIC | Serous cystadenocarcinoma | CA125-510 units/mL |
| 3 | 62 | T3N0M0, stage IIB | Serous cystadenocarcinoma | CA125-120 units/mL |
| 4 | 56 | T2N0M0, stage IIB | Endometrioid | CA125-350 units/mL |
| | | | cystadenocarcinoma | |
| 5 | 64 | T1N0M0, stage IA | serous-mucinous | CA125-240 units/mL |
| | | | cystadenocarcinoma | |
| 6 | 67 | T2N0M0, stage IIB | Serous cystadenocarcinoma | CA125-370 units/mL |
| 7 | 53 | T3N1M0, stage IIIC | Serous cystadenocarcinoma | CA125-600 units/mL |
| 8 | 46 | T2N0M0, stage IIB | granulosa cell cancer | CA125-350 units/mL |
| 9 | 30 | T2N0M0, stage IIB | Endometrioid | CA125-540 units/mL |
| | | | cystadenocarcinoma | |
| 10 | 63 | T1N0M0, stage IA | Serous cystadenocarcinoma | CA125-120 units/mL |
| 11 | 50 | T2N1M0, stage IIIC | Serous cystadenocarcinoma | CA125-200 units/mL |

Melanoma

| No | Age | Diagnosis | Biopsy |
|---|---|---|---|
| 1 | 71 | Melanoma of the skin right foot, | Epithelioid melanoma, Clark's level 5. |
| | | T4N0M0, stage III | Breslow's depth 12 MM |
| 2 | 67 | Melanoma of abdominal skin, | Epithelioid melanoma, Clark's level 5. |
| | | T4N2M0, stage III | Breslow's depth 20 MM |
| 3 | 68 | Melanoma of the skin of the right | Epithelioid melanoma, Clark's level 5. |
| | | shoulder, T4N1M0, stage IIIB | Breslow's depth 10 MM |
| 4 | 38 | Melanoma skin left foot, T4N0M0, | Epithelioid melanoma, Clark's level 4. |
| | | stage III | Breslow's depth 8 MM |
| 5 | 60 | Melanoma skin left chest T2N0M0, | Epithelioid melanoma, Clark's level 2. |
| | | stage II | Breslow's depth 4 MM |
| 6 | 78 | Melanoma of the left hand and | Epithelioid melanoma, Clark's level 4. |
| | | forearm skin, T3N0M0, stage III | Breslow's depth 10 MM |
| 7 | 60 | Melanoma of the skin area to the | Epithelioid melanoma, Clark's level 5. |
| | | left subscapular, T3N0M0, stage III | Breslow's depth 11 MM |
| 8 | 72 | Melanoma skin nasolabial triangle, | Epithelioid melanoma, Clark's level 2. |
| | | T2N2M0, stage II | Breslow's depth 4 MM |
| 9 | 48 | Melanoma of abdominal skin, | Epithelioid melanoma, Clark's level 4. |
| | | T4N2M0, stage IV | Breslow's depth 14 MM |
| 10 | 56 | Melanoma of abdominal skin, | Epithelioid melanoma, Clark's level 5. |
| | | T4N2M0, stage III | Breslow's depth 12 MM |
| 11 | 58 | Melanoma skin armpit, T4N2M0, | Epithelioid melanoma, Clark's level 5. |
| | | stage III | Breslow's depth 10 MM |
| 12 | 63 | Melanoma of the skin of the neck | Epithelioid melanoma, Clark's level 5. |
| | | occipital T4N1M0, stage III | Breslow's depth 15 MM |
| 13 | 71 | Melanoma of abdominal skin, | Epithelioid melanoma, Clark's level 5. |
| | | T4N2M0, stage III | Breslow's depth 18 MM |
| 14 | 43 | Melanoma skin left chest T2N0M0, | Epithelioid melanoma, Clark's level 3. |
| | | stage III | Breslow's depth 6 MM |

The results of using various antigens in the assay system for identification of autoantibodies such as IgG and IgA were shown.

### EXAMPLES

### Example 1

### Detection of IgG and IgA Autoantibodies in Prostate Cancer

Diagnoses of patients with prostate cancer were established on the basis of the following indicators: clinical examination with morphological confirmation of the diagnosis and on the basis of cancer markers (PSA). Only in this group included 30 patients.

Immunoassay test (ELISA) of samples taken from prostate cancer patients and a control sample was made according to the procedure described. Considered positive samples had an optical density in the ELISA by 20% or more of the optical density of the control sample.

### Results:

When used as a Glu-plasminogen antigen in ELISA, the number of positive samples in prostate cancer patients was 83% for IgG and 86% for IgA.

When used as an antigen heavy chain (Glu1-Arg561) in ELISA, the number of p itive samples in prostate cancer patients was 83% for IgG and 80% for IgA.

When used as an antigen light chain (Val562-Asn791 in ELISA, the number of positive samples in prostate cancer patients was 57% for IgG and 63% for IgA.

When used as an antigen miniplasminogen (Val442-Asn791) in ELISA, the number of positive samples in prostate cancer patients was 69% for IgG and 68% for IgA.

When used as an antigen fragment K1-4, 5 (Lys78-Arg530) in ELISA, the number of positive samples in prostate cancer patients was 83% for IgG and 74% for IgA.

When used as an antigen fragment K1-4 (Tyr80-Asn440) in ELISA, the number of positive samples in prostate cancer patients was 80% for IgG and 74% for IgA

When used as an antigen fragment K1-3 (Tyr80-Val338) in ELISA, the number of positive samples in prostate cancer patients was 77% for IgG and 69% for IgA.

When used as an antigen fragment K2-3 (Cys165-Val338) in ELISA, the number of positive samples in prostate cancer patients was 61% for IgG and 56% for IgA.

When used as an antigen fragment K4-5 (Val355-Phe546) in ELISA, the number of positive samples in prostate cancer patients was 60% for IgG and 51% for IgA.

When used as an antigen fragment K1 (Tur80-Glu164) in ELISA, the number of positive samples in prostate cancer patients was 45% for IgG and 39% for IgA.

When used as an antigen fragment K4 (Val354-Ala439) in ELISA, the number of positive samples in prostate cancer patients was 44% for IgG and 30% for IgA.

When used as an antigen fragment K5 (Ser441-Fhe546) in ELISA, the number of positive samples in prostate cancer patients was 56% for IgG and 36% for IgA.

### Example 2

### Diagnosis of Lung Cancer

Diagnosis of patients with lung cancer have been established on the basis of a clinical study with morphological confirmation of the diagnosis. Only in this group included 20 patients.

Immunoassay test (ELISA) of samples taken from patients with lung cancer and a control sample was made according to the procedure described. Considered positive samples had an optical density in the ELISA by 20% or more of the optical density of the control sample.

### Results.

When used as a Glu-plasminogen antigen in ELISA, the number of positive samples with lung cancer patients was 80% for IgG and 55% for IgA.

When used as an antigen heavy chain (Glu1-Arg561) in ELISA, the number of positive samples in patients with lung cancer was 76% for IgG and 65% for IgA.

When used as an antigen light chain (Val562-Asn791) in ELISA, the number of positive samples in patients with lung cancer was 45% for IgG and 75% for IgA.

When used as an antigen miniplasminogen (Val442-Asn791) in ELISA, the number of positive samples in patients with lung cancer was 55% for IgG and 75% for IgA.

When used as an antigen fragment K1-4, 5 (Lys78-Arg530) in ELISA, the number of positive samples in patients with lung cancer was 76% for IgG and 75% for IgA.

When used as an antigen fragment K1-4 (Tyr80-Asn440) in ELISA, the number of positive samples in patients with lung cancer was 72% for IgG and 75% for IgA.

When used as an antigen fragment K1-4 (Asn60-Pro447) in ELISA, the number of positive samples in patients with lung cancer was 45% for IgG and 75% for IgA.

When used as an antigen fragment K1-4 (Lys78-Pro447) in ELISA, the number of positive samples in patients with lung cancer was 77% for IgG and 75% for IgA.

When used as an antigen fragment K1-4 (Lys78-Pro446) in ELISA, the number of positive samples in patients with lung cancer was 77% for IgG and 75% for IgA.

When used as an antigen fragment K1-4 (Lys78-Lys468) in ELISA, the number of positive samples in patients with lung cancer was 81% for IgG and 79% for IgA.

When used as an antigen fragment K1-4 (Leu74-Leu451) in ELISA, the number of positive samples in patients with lung cancer was 81% for IgG and 75% for IgA.

When used as an antigen fragment K1-3 (Tyr80-Val338) in ELISA, the number of positive samples in patients with lung cancer was 68% for IgG and 65% for IgA.

When used as an antigen fragment K1-3 (Tyr80-Val354) in ELISA, the number of positive samples in patients with lung cancer was 68% for IgG and 65% for IgA.

When used as an antigen fragment K1-3 (Leu74-Pro349) in ELISA, the number of positive samples in patients with lung cancer was 68% for IgG and 65% for IgA.

When used as an antigen fragment K2-3 (Cys165-Val354) in ELISA, the number of positive samples in patients with lung cancer was 45% for IgG and 60% for IgA.

When used as an antigen fragment K4-5 (Val355-Phe546) in ELISA, the number of positive samples in patients with lung cancer was 35% for IgG and 55% fo in ELISA r IgA.

When used as an antigen fragment K1 (Tur80-Glu164) in ELISA, the number of positive samples in patients with lung cancer was 35% for IgG and 45% for IgA.

When used as an antigen fragment K4 (Val354-Ala439) in ELISA, the number of positive samples in patients with lung cancer was 25% for IgG and 40% for IgA.

When used as an antigen fragment K5 (Ser441-Fhe546) in ELISA, the number of positive samples in patients with lung cancer was 34% for IgG and 35% for IgA.

### Example 3

### Diagnosis of Breast Cancer

Diagnoses of patients with breast cancer have been established on the basis of clinical examination with morphological confirmation of the diagnosis and on the basis of cancer markers (CA 15-3). Only in this group included 40 patients.

Immunoassay test (ELISA) of samples taken from breast cancer patients and a control sample was made according to the procedure described. Considered positive samples had an optical density in the ELISA by 20% or more of the optical density of the control sample.

### Results.

When used as a Glu-plasminogen as an antigen in ELISA, the number of positive samples in patients with breast cancer was 63% for IgG and 53% for IgA.

When used as an antigen heavy chain (Glu1-Arg561) in ELISA, the number of positive samples in patients with breast cancer was 65% for IgG and 65% for IgA.

When used as an antigen light chain (Val562-Asn791) in ELISA, the number of positive samples in patients with breast cancer was 50% for IgG and 60% for IgA.

When used as an antigen miniplasminogen (Val442-Asn791) in ELISA, the number of positive samples in patients with breast cancer was 60% for IgG and 65% for IgA.

When used as an antigen fragment K1-4, 5 (Lys78-Arg530) in ELISA, the number of positive samples in patients with breast cancer was 66% for IgG and 65% for IgA.

When used as an antigen fragment K1-4 (Tyr80-Asn440) in ELISA, the number of positive samples in patients with breast cancer was 67% for IgG and 65% for IgA.

When used as an antigen fragment K1-3 (Tyr80-Val338) in ELISA, the number of positive samples in patients with breast cancer was 62% for IgG and 60% for IgA.

When used as an antigen fragment K2-3 (Cys165-Val338) in ELISA, the number of positive samples in patients with breast cancer was 45% for IgG and 50% for IgA.

When used as an antigen fragment K4-5 (Val355-Phe546) in ELISA, the number of positive samples in patients with breast cancer was 40% for IgG and 45% for IgA.

When used as an antigen fragment K1 (Tur80-Glu164) in ELISA, the number of positive samples in patients with breast cancer was 35% for IgG and 35% for IgA.

When used as an antigen fragment K4 (Val354-Ala439) in ELISA, the number of positive samples in patients with breast cancer was 25% for IgG and 30% for IgA.

When used as an antigen fragment K5 (Ser441-Fhe546) in ELISA, the number of positive samples in patients with breast cancer was 25% for IgG and 30% for IgA.

### Example 4

### Diagnosis of Ovarian Cancer

Diagnoses of patients with ovarian cancer were established on the basis of the following indicators: clinical examination with morphological confirmation of the diagnosis and on the basis of cancer markers (CA 125). Only in this group had 11 patients.

Immunoassay test (ELISA) of samples from patients with ovarian cancer, and a control sample was made according to the procedure described. Considered positive samples had an optical density in the ELISA by 20% or more of the optical density of the control sample

### Results.

When used as a Glu-plasminogen as an antigen in ELISA, the number of positive samples from patients with ovarian cancer was 73% for IgG and 64% for IgA.

When used as an antigen heavy chain (Glu1-Arg561) in ELISA, the number of positive samples from patients with ovarian cancer was 71% for IgG and 57% for IgA.

When used as an antigen light chain (Val562-Asn791) in ELISA, the number of positive samples from patients with ovarian cancer was 50% for IgG and 45% for IgA.

When used as an antigen miniplasminogen (Val442-Asn791) in ELISA, the number of positive samples from patients with ovarian cancer was 55% for IgG and 45% for IgA.

When used as an antigen fragment K1-4, 5 (Lys78-Arg530) in ELISA, the number of positive samples from patients with ovarian cancer was 64% for IgG and 45% for IgA.

When used as an antigen fragment K1-4 (Tyr80-Asn440) in ELISA, the number of positive samples from patients with ovarian cancer was 64% for IgG and 45% for IgA

When used as an antigen fragment K1-3 (Tyr80-Val338) in ELISA, the number of positive samples from patients with ovarian cancer was 54% for IgG and 36% for IgA.

When used as an antigen fragment K2-3 (Cys165-Val338) in ELISA, the number of positive samples from patients with ovarian cancer was 45% for IgG and 36% for IgA.

When used as an antigen fragment K4-5 (Val355-Phe546) in ELISA, the number of positive samples from patients with ovarian cancer was 45% for IgG and 36% for IgA.

When used as an antigen fragment K1 (Tur80-Glu164) in ELISA, the number of positive samples from patients with ovarian cancer was 27% for IgG and 27% for IgA.

When used as an antigen fragment K4 (Val354-Ala439) in ELISA, the number of positive samples from patients with ovarian cancer was 27% for IgG and 27% for IgA.

When used as an antigen fragment K5 (Ser441-Fhe546) in ELISA, the number of positive samples from patients with ovarian cancer was 27% for IgG and 36% for IgA.

### Example 5

### Melanoma

Diagnoses of patients with melanoma have been confirmed on the basis of: clinical examination with morphological confirmation of the diagnosis. Only in this group included 14 patients. Immunoassay test (ELISA) of samples taken from patients with melanoma and a control sample was made according to the procedure described. Considered positive samples had an optical density in the ELISA by 20% or more of the optical density of the control sample.

### Results.

When used as a Glu-plasminogen as an antigen in ELISA, the number of positive samples in patients with melanoma, 78% for IgG and 37% for IgA.

When used as an antigen heavy chain (Glu1-Arg561) in ELISA, the number of positive samples in patients with melanoma, 78% for IgG and 37% for IgA.

When used as an antigen light chain (Val562-Asn791) in ELISA, the number of positive samples from melanoma patients was 62% for IgG and 43% for IgA.

When used as an antigen minipiasminogen (Val442-Asn791) in IFA, the number of positive samples from melanoma patients was 62% for IgG and 43% for IgA.

When used as an antigen fragment K1-4, 5 (Lys78-Arg530) in ELISA, the number of positive samples from melanoma patients was 72% for IgG and 37% for IgA.

When used as an antigen fragment K1-4 (Tyr80-Asn440) in ELISA, the number of positive samples from melanoma patients was 72% for IgG and 37% for IgA.

When used as an antigen fragment K1-3 (Tyr80-Val338) in ELISA, the number of positive samples from melanoma patients was 65% for IgG and 35% for IgA.

When used as an antigen fragment K2-3 (Cys165-Val338) in ELISA, the number of positive samples from melanoma patients was 57% for IgG and 35% for IgA.

When used as an antigen fragment K4-5 (Val355-Phe546) in ELISA, the number of positive samples from melanoma patients was 57% for IgG and 35% for IgA.

When used as an antigen fragment K1 (Tur80-Glu164) in ELISA, the number of positive samples from melanoma patients was 35% for IgG and 29% for IgA.

When used as an antigen fragment K4 (Val354-Ala439) in ELISA, the number of positive samples from melanoma patients was 35% for IgG and 29% for IgA.

When used as an antigen fragment K5 (Ser441-Fhe546) in ELISA, the number of positive samples from melanoma patients was 35% for IgG and 29% for IgA.

### Sequence Listing

<110> Goufman Evgeny Iosifovich, Iakovlev Vasily Nikolaevich, Kanaev Alexey Alexeevich, Suleymanov Rustam Raisovich.
<120> Method for the diagnosis of cancer and enzyme-linked immunoassay (ELISA) kit for its application.

<160> Number sequences-20
<210> 1
<212> Protein
<213> Homo sapiens
<400> Description of the sequence
SEQ ID NO: 1
Glu¹ Pro Leu Asp Asp Tyr Val Asn Thr Gln¹⁰ Gly Ala Ser Leu Phe Ser Val Thr Lys Lys²⁰ Gln Leu
Gly Ala Gly Ser Ile Glu Glu Cys³⁰ Ala Ala Lys Cys Glu Glu Asp Glu Glu Phe⁴⁰ Thr Cys Arg Ala
Phe Gln Tyr His Ser Lys⁵⁰ Glu Gln Gln Cys Val Ile Met Ala Glu Asn⁶⁰ Arg Lys Ser Ser Ile Ile Ile
Arg Met Arg⁷⁰ Asp Val Val Leu Phe Glu Lys Lys⁷⁸ Val Tyr⁸⁰ Leu Ser Glu Cys Lys Thr Gly Asn
Gly Lys⁹⁰ Asn Tyr Arg Gly Thr Met Ser Lys Thr Lys¹⁰⁰ Asn Gly Ile Thr Cys Gln Lys Trp Ser
Ser¹¹⁰ Thr Ser Pro His Arg Pro Arg Phe Ser Pro¹²⁰ Ala Thr His Pro Ser Glu Gly Leu Glu Glu¹³⁰
Asn Tyr Cys Arg Asn Pro Asp Asn Asp Pro¹⁴⁰ Gln Gly Pro Trp Cys Tyr Thr Thr Asp Pro¹⁵⁰ Glu
Lys Arg Tyr Asp Tyr Cys Asp Ile Leu¹⁶⁰ Glu Cys Glu Glu Glu Cys Met His Cys Ser¹⁷⁰ Gly Glu
Asn Tyr Asp Gly Lys Ile Ser Lys¹⁸⁰ Thr Met Ser Gly Leu Glu Cys Gln Ala Trp⁸⁰ Asp Ser Gln Ser
Pro His Ala His Gly Tyr²⁰⁰ Ile Pro Ser Lys Phe Pro Asn Lys Asn Leu²¹⁰ Lys Lys Asn Tyr Cys Arg
Asn Pro Asp Arg²²⁰ Glu Leu Arg Pro Trp Cys Phe Thr Thr Asp²³⁰ Pro Asn Lys Arg Trp Glu Leu
Cys Asp Ile²⁴⁰ Pro Arg Cys Thr Thr Pro Pro Pro Ser Ser²⁵⁰ Gly Pro Thr Tyr Gln Cys Leu Lys Gly
Thr²⁶⁰ Gly Glu Asn Tyr Arg Gly Asn Val Ala Val Thr Val Ser Gly His Thr Cys Gln His Trp²⁸⁰ Ser
Ala Gln Thr Pro His Thr His Asn Arg²⁹⁰ Thr Pro Glu Asn Phe Pro Cys Lys An Leu³⁰⁰ Asp Glu
Asn Tyr Cys Arg Asn Pro Asp Gly³¹⁰ Lys Arg Ala Pro Trp Cys His Thr Thr Asn³²⁰ Ser Gln Val
Arg Trp Glu Tyr Cys Lys Ile³³⁰ Pro Ser Cys Asp Ser Ser Pro Val Ser Thr³⁴⁰ Glu Gln Leu Ala Pro
Thr Ala Pro Pro Glu³⁵⁰ Leu Thr Pro Val Val Gln Asp Cys Tyr His³⁶⁰ Gly Asp Gly Gln Ser Tyr Arg
Gly Thr Ser³⁷⁰ Ser Thr Thr Thr Thr Gly Lys Lys Cys Gln³⁸⁰ Ser Trp Ser Ser Met Thr Pro His Arg
His³⁹⁰ Gln Lys Thr Pro Glu Asn Tyr Pro Asn Ala⁴⁰⁰ Gly Leu Thr Met Asn Tyr Cys Arg Asn Pro⁴¹⁰
Asp Ala Asp Lys Gly Pro Trp Cys Phe Thr⁴²⁰ Thr Asp Pro Ser Val Arg Trp Glu Tyr Cys⁴³⁰ Asn
Leu Lys Lys Cys Ser Gly Thr Glu Ala⁴⁴⁰ Ser Val Val Ala Pro Pro Pro Val Val Leu⁴⁵⁰ Leu Pro Asp
Val Glu Thr Pro Ser Glu Glu⁴⁶⁰ Asp Cys Met Phe Gly Asn Gly Lys Gly Tyr⁴⁷⁰ Arg Gly Lys Aug
Ala Thr Thr Val Thr Gly⁴⁸⁰ Thr Pro Cys Gln Asp Trp Ala Ala Gln Glu⁴⁹⁰ Pro His Arg His Ser Ile
Phe Thr Pro Glu⁵⁰⁰ Thr Asn Pro Arg Ala Gly Leu Glu Lys Asn⁵¹⁰ Tyr Cys Arg Asn Pro Asp Gly
Asp Val Gly⁵²⁰ Gly Pro Trp Cys Tyr Thr Thr Asn Pro Arg⁵³⁰ Lys Leu Tyr Asp Tyr Cys Asp Val
Pro Gln⁵⁴⁰ Cys Ala Ala Pro Ser Phe Asp Cys Gly Lys⁵⁵⁰ Pro Gln Val Glu Pro Lys Lys Cys Pro
Gly Arg Val Val Gly Gly Cys Val Ala His Pro⁵⁷⁰ His Ser Trp Pro Trp Gln Val Ser Leu Arg⁵⁸⁰
Thr Aug Phe Gly Met His Phe Cys Gly Gly⁵⁹⁰ Thr Leu Ile Ser Pro Glu Trp Val Leu Thr⁶⁰⁰ Ala Ala
His Cys Leu Glu Lys Ser Pro Arg⁶¹⁰ Pro Ser Ser Tyr Lys Val Ile Ley Gly Ala⁶²⁰ His Gln Glu Val
Asn Leu Glu Pro His Val⁶³⁰ Gln Glu Ile Glu Val Ser Aug Leu Phe Leu⁶⁴⁰ Glu Pro Thr Aug Lys
Asp Ile Ala Leu Leu⁶⁵⁰ Lys Leu Ser Ser Pro Ala Val Ile Thr Asp⁶⁶⁰ Lys Val Ile Pro Ala Cys Leu
Pro Ser Pro⁶⁷⁰ Asn Tyr Val Val Ala Asp Aug Thr Glu Cys⁶⁸⁰ Phe Ile Thr Gly Trp Gly Glu Thr Gln
Gly⁶⁹⁰ Thr Phe Gly Ala Gly Leu Leu Lys Glu Ala⁷⁰⁰ Gln Leu Pro Val Ile Glu Asn Lys Val Cys⁷¹⁰
Asn Arg Tyr Glu Phe Leu Asn Gly Arg Va⁷²⁰l Gln Ser Thr Glu Leu Cys Ala Gly His Lue⁷³⁰ Ala
Gly Gly Thr Asp Ser Cys Gln Gly Asp⁷⁴⁰ Ser Gly Gly Pro Leu Val Cys Phe Glu Lys⁷⁵⁰ Asp Lys Tyr
Ile Leu Gln Gly Val Thr Ser⁷⁶⁰ Trp Gly Leu Gly Cys Ala Aug Pro Asn Lys⁷⁷⁰ Pro Gly Val Tyr Val
Aug Val Ser Aug Phe⁷⁸⁰ Val Thr Trp Ile Glu Gly Val Met Arg Asⁿ⁷⁹⁰ Asn
<210> 2
<212> Protein
<213> Homo sapiens
<400> Description of the sequence
SEQ ID NO: 2
Lys⁷⁸ Val Tyr⁸⁰ Leu Ser Glu Cys Lys Thr Gly Asn Gly Lys⁹⁰ Asn Tyr Aug Gly Thr Met Ser Lys Thr
Lys¹⁰⁰ Asn Gly Ile Thr Cys Gln Lys Trp Ser Ser¹¹⁰ Thr Ser Pro His Aug Pro Aug Phe Ser Pro¹²⁰
Ala Thr His Pro Ser Glu Gly Leu Glu Glu¹³⁰ Asn Tyr Cys Arg Asn Pro Asp Asn Asp Pro¹⁴⁰ Gln
Gly Pro Trp Cys Tyr Thr Thr Asp Pro¹⁵⁰ Glu Lys Aug Tyr Asp Tyr Cys Asp Ile Leu¹⁶⁰ Glu Cys Glu
Glu Glu Cys Met His Cys Ser¹⁷⁰ Gly Glu Ash Tyr Asp Gly Lys Ile Ser Lys¹⁸⁰ Thr Met Ser Gly Leu
Glu Cys Gln Ala Trp⁸⁰ Asp Ser Gln Ser Pro His Ala His Gly Tyr²⁰⁰ Ile Pro Ser Lys Phe Pro Asn
Lys Asn Leu²¹⁰ Lys Lys Asn Tyr Cys Arg Asn Pro Asp Arg²²⁰ Glu Leu Arg Pro Trp Cys Phe Thr
Thr Asp²³⁰ Pro Asn Lys Arg Trp Glu Leu Cys Asp Ile²⁴⁰ Pro Aug Cys Thr Thr Pro Pro Pro Ser
Ser²⁵⁰ Gly Pro Thr Tyr Gln Cys Leu Lys Gly Thr²⁶⁰ Gly Glu Asn Tyr Arg Gly Asn Val Ala Val Thr
Val Ser Gly His Thr Cys Gln His Trp²⁸⁰ Ser Ala Gln Thr Pro His Thr His Asn Arg²⁹⁰ Thr Pro Glu
Asn Phe Pro Cys Lys Asn Leu³⁰⁰ Asp Glu Asn Tyr Cys Arg Asn Pro Asp Gly³¹⁰ Lys Arg Ala Pro
Trp Cys His Thr Thr Asn³²⁰ Ser Gln Val Arg Trp Glu Tyr Cys Lys Ile³³⁰ Pro Ser Cys Asp Ser Ser
Pro Val Ser Thr³⁴⁰ Glu Gln Leu Ala Pro Thr Ala Pro Pro Glu³⁵⁰ Leu Thr Pro Val Val Gln Asp Cys
Tyr His³⁶⁰ Gly Asp Gly Gln Ser Tyr Arg Gly Thr Ser³⁷⁰ Ser Thr Thr Thr Thr Gly Lys Lys Cys
Gln³⁸⁰ Ser Trp Ser Ser Met Thr Pro His Arg His³⁹⁰ Gln Lys Thr Pro Glu Asn Tyr Pro Asn Ala⁴⁰⁰
Gly Leu Thr Met Asn Tyr Cys Arg Asn Pro⁴¹⁰ Asp Ala Asp Lys Gly Pro Trp Cys Phe Thr⁴²⁰ Thr
Asp Pro Ser Val Arg Trp Glu Tyr Cys⁴³⁰ Asn Leu Lys Lys Cys Ser Gly Thr Glu Ala⁴⁴⁰ Ser Val Val
Ala Pro Pro Pro Val Val Leu⁴⁵⁰ Leu Pro Asp Val Glu Thr Pro Ser Glu Glu⁴⁶⁰ Asp Cys Met Phe
Gly Asn Gly Lys Gly Tyr⁴⁷⁰ Arg Gly Lys Arg Ala Thr Thr Val Thr Gly⁴⁸⁰ Thr Pro Cys Gln Asp Trp
Ala Ala Gln Glu⁴⁹⁰ Pro His Arg His Ser Ile Phe Thr Pro Glu⁵⁰⁰ Thr Asn Pro Arg Ala Gly Leu Glu
Lys Asn⁵¹⁰ Tyr Cys Arg Asn Pro Asp Gly Asp Val Gly⁵²⁰ Gly Pro Trp Cys Tyr Thr Thr Asn Pro
Arg⁵³⁰ Lys Leu Tyr Asp Tyr Cys Asp Val Pro Gln⁵⁴⁰ Cys Ala Ala Pro Ser Phe Asp Cys Gly Lys⁵⁵⁰
Pro Gln Val Glu Pro Lys Lys Cys Pro Gly⁵⁶⁰ Arg Val Val Gly Gly Cys Val Ala His Pro⁵⁷⁰ His Ser
Trp Pro Trp Gln Val Ser Leu Arg⁵⁸⁰ Thr Arg Phe Gly Met His Phe Cys Gly Gly⁵⁹⁰ Thr Leu Ile
Ser Pro Glu Trp Val Leu Thr⁶⁰⁰ Ala Ala His Cys Leu Glu Lys Ser Pro Arg⁶¹⁰ Pro Ser Ser Tyr Lys
Val Ile Leu Gly Ala⁶²⁰ His Gln Glu Val Asn Leu Glu Pro His Val⁶³⁰ Gln Glu Ile Glu Val Ser Arg
Leu Phe Leu⁶⁴⁰ Glu Pro Thr Arg Lys Asp Ile Ala Leu Leu⁶⁵⁰ Lys Leu Ser Ser Pro Ala Val Ile Thr
Asp⁶⁶⁰ Lys Val Ile Pro Ala Cys Leu Pro Ser Pro⁶⁷⁰ Asn Tyr Val Val Asp Arg Thr Glu Cys⁶⁸⁰
Phe Ile Thr Gly Trp Gly Glu Thr Gln Gly⁶⁹⁰ Thr Phe Gly Ala Gly Leu Leu Lys Glu Ala⁷⁰⁰ Gln Leu
Pro Val Ile Glu Asn Lys Val Cys⁷¹⁰ Asn Arg Tyr Glu Phe Leu Asn Gly Arg Va⁷²⁰l Gln Ser Thr Glu
Leu Cys Ala Gly His Leu⁷³⁰ Ala Gly Gly Thr Asp Ser Cys Gln Gly Asp⁷⁴⁰ Ser Gly Gly Pro Leu
Val Cys Phe Glu Lys⁷⁵⁰ Asp Lys Tyr Ile Leu Gln Gly Val Thr Ser⁷⁶⁰ Trp Gly Leu Gly Cys Ala Arg
Pro Asn Lys⁷⁷⁰ Pro Gly Val Tyr Val Arg Val Ser Arg Phe⁷⁸⁰ Val Thr Trp Ile Glu Gly Val Met Arg
Asn⁷⁹⁰ Asn
<210> 3
<212> protein
<213> Homo sapiens
<400> Description of the sequence
SEQ ID NO: 3
Glu¹ Pro Leu Asp Asp Tyr Val Asn Thr Gln¹⁰ Gly Ala Ser Leu Phe Ser Val Thr Lys Lys²⁰ Gln Leu
Gly Ala Gly Ser Ile Glu Glu Cys³⁰ Ala Ala Lys Cys Glu Glu Asp Glu Glu Phe⁴⁰ Thr Cys Arg Ala
Phe Gln Tyr His Ser Lys⁵⁰ Glu Gln Gln Cys Val Ile Met Ala Glu Asn⁶⁰ Arg Lys Ser Ser Ile Ile Ile
Arg Met Arg⁷⁰ Asp Val Val Leu Phe Glu Lys Lys⁷⁸ Val Tyr⁸⁰ Leu Ser Glu Cyc Lys Thr Gly Asn
Gly Lys⁹⁰ Asn Tyr Arg Gly Thr Met Ser Lys Thr Lys¹⁰⁰ Asn Gly Ile Thr Cys Gln Lys Trp Ser
Ser¹¹⁰ Thr Ser Pro His Arg Pro Arg Phe Ser Pro¹²⁰ Ala Thr His Pro Ser Glu Gly Leu Glu Glu¹³⁰
Asn Tyr Cys Arg Asn Pro Asp Asn Asp Pro¹⁴⁰ Gln Gly Pro Trp Cys Tyr Thr Thr Asp Pro¹⁵⁰ Glu
Lys Arg Tyr Asp Tyr Cys Asp Ile Leu¹⁶⁰ Glu Cys Glu Glu Glu Cys Met His Cys Ser¹⁷⁰ Gly Glu
Asn Tyr Asp Gly Lys Ile Ser Lys¹⁸⁰ Thr Met Ser Gly Leu Glu Cys Gln Ala Trp⁸⁰ Asp Ser Gln Ser
Pro His Ala His Gly Tyr²⁰⁰ Ile Pro Ser Lys Phe Pro Asn Lys Asn Leu²¹⁰ Lys Lys Asn Tyr Cys Arg
Asn Pro Asp Arg²²⁰ Glu Leu Arg Pro Trp Cys Phe Thr Thr Asp²³⁰ Pro Asn Lys Arg Trp Glu Leu
Cys Asp Ile²⁴⁰ Pro Arg Cys Thr Thr Pro Pro Pro Ser Ser²⁵⁰ Gly Pro Thr Tyr Gln Cys Leu Lys Gly
Thr²⁶⁰ Gly Glu Asn Tyr Arg Gly Asn Val Ala Val Thr Val Ser Gly His Thr Cys Gln His Trp²⁸⁰ Ser
Ala Gln Thr Pro His Thr His Asn Arg²⁹⁰ Thr Pro Glu Asn Phe Pro Cys Lys Asn Leu³⁰⁰ Asp Glu
Asn Tyr Cys Arg Asn Pro Asp Gly³¹⁰ Lys Arg Ala Pro Trp Cys His Thr Thr Asn³²⁰ Ser Gln Val
Arg Trp Glu Tyr Cys Lys Ile³³⁰ Pro Ser Cys Asp Ser Ser Pro Val Ser Thr³⁴⁰ Glu Gln Leu Ala Pro
Thr Ala Pro Pro Glu³⁵⁰ Leu Thr Pro Val Val Gln Asp Cys Tyr His³⁶⁰ Gly Asp Gly Gln Ser Tyr Arg
Gly Thr Ser³⁷⁰ Ser Thr Thr Thr Thr Gly Lys Lys Cys Gln³⁸0 Ser Trp Ser Ser Met Thr Pro His Arg
His³⁹⁰ Gln Lys Thr Pro Glu Asn Tyr Pro Asn Ala⁴⁰⁰ Gly Leu Thr Met Asn Tyr Cys Arg Asn Pro⁴¹⁰
Asp Ala Asp Lys Gly Pro Trp Cys Phe Thr⁴²⁰ Thr Asp Pro Ser Val Arg Trp Glu Tyr Cys⁴³⁰ Asn
Leu Lys Lys Cys Ser Gly Thr Glu Ala⁴⁴⁰ Ser Val Val Ala Pro Pro Pro Val Val Leu⁴⁵⁰ Leu Pro Asp
Val Glu Thr Pro Ser Glu Glu⁴⁶⁰ Asp Cys Met Phe Gly Asn Gly Lys Gly Tyr⁴⁷⁰ Arg Gly Lys Arg
Ala Thr Thr Val Thr Gly⁴⁸⁰ Thr Pro Cys Gln Asp Trp Ala Ala Gln Glu⁴⁹⁰ Pro His Arg His Ser Ile
Phe Thr Pro Glu⁵⁰⁰ Thr Asn Pro Arg Ala Gly Leu Glu Lys Asn⁵¹⁰ Tyr Cys Arg Asn Pro Asp Gly
Asp Val Gly⁵²⁰ Gly Pro Trp Cys Tyr Thr Thr Asn Pro Arg⁵³⁰ Lys Leu Tyr Asp Tyr Cys Asp Val
Pro Gln⁵⁴⁰ Cys Ala Ala Pro Ser Phe Asp Cys Gly Lys⁵⁵⁰ Pro Gln Val Glu Pro Lys Lys Cys Pro
Gly⁵⁶⁰ Arg
<210> 3
<212> protein
<213> Homo sapiens
<400> Description of the sequence
SEQ ID NO: 4
Lys⁷⁸ Val Tyr⁸⁰ Leu Ser Glu Cys Lys Thr Gly Asn Gly Lys⁹⁰ Asn Tyr Arg Gly Thr Met Ser Lys Thr
Lys¹⁰⁰ Asn Gly Ile Thr Cys Gln Lys Trp Ser Ser¹¹⁰ Thr Ser Pro His Arg Pro Arg Phe Ser Pro¹²⁰
Ala Thr His Pro Set Glu Gly Leu Glu Glu¹³⁰ Asn Tyr Cys Arg Asn Pro Asp Asn Asp Pro¹⁴⁰ Gln
Gly Pro Trp Cys Tyr Thr Thr Asp Pro¹⁵⁰ Glu Lys Arg Tyr Asp Tyr Cys Asp Ile Leu¹⁶⁰ Glu Cys Glu
Glu Glu Cys Met His Cys Ser¹⁷⁰ Gly Glu Asn Tyr Asp Gly Lys Ile Ser Lys¹⁸⁰ Thr Met Ser Gly Leu
Glu Cys Gln Ala Trp⁸⁰ Asp Ser Gln Ser Pro His Ala His Gly Tyr²⁰⁰ Ile Pro Ser Lys Phe Pro Asn
Lys Asn Leu²¹⁰ Lys Lys Asn Tyr Cys Arg Asn Pro Asp Arg²²⁰ Glu Leu Arg Pro Trp Cys Phe Thr
Thr Asp²³⁰ Pro Asn Lys Arg Trp Glu Leu Cys Asp Ile²⁴⁰ Pro Arg Cys Thr Thr Pro Pro Pro Ser
Ser²⁵⁰ Gly Pro Thr Tyr Gln Cys Leu Lys Gly Thr²⁶⁰ Gly Glu Asn Tyr Arg Gly Asn Val Ala Val Thr
Val Ser Gly His Thr Cys Gln His Trp²⁸⁰ Ser Ala Gln Thr Pro His Thr His Asn Arg²⁹⁰ Thr Pro Glu
Asn Phe Pro Cys Lys Asn Leu³⁰⁰ Asp Glu Asn Tyr Cys Arg Asn Pro Asp Gly³¹⁰ Lys Arg Ala Pro
Trp Cys His Thr Thr Asn³²⁰ Ser Gln Val Arg Trp Glu Tyr Cys Lys Ile³³⁰ Pro Ser Cys Asp Ser Ser
Pro Val Ser Thr³⁴⁰ Glu Gln Leu Ala Pro Thr Ala Pro Pro Glu³⁵⁰ Leu Thr Pro Val Val Gln Asp Cys
Tyr His³⁶⁰ Gly Asp Gly Gln Ser Tyr Arg Gly Thr Ser³⁷⁰ Ser Thr Thr Thr Thr Gly Lys Lys Cys
Gle³⁸⁰ Ser Trp Ser Ser Met Thr Pro His Arg His³⁹⁰ Gln Lys Thr Pro Glu Asn Tyr Pro Asn Ala400
Gly Leu Thr Met Asn Tyr Cys Arg Asn Pro⁴¹⁰ Asp Ala Asp Lys Gly Pro Trp Cys Phe Thr⁴²⁰ Thr
Asp Pro Ser Val Arg Trp Glu Tyr Cys⁴³⁰ Asn Leu Lys Lys Cys Ser Gly Thr Glu Ala⁴⁴⁰ Ser Val Val
Ala Pro Pro Pro Val Val Leu⁴⁵⁰ Leu Pro Asp Val Glu Thr Pro Ser Glu Glu⁴⁶⁰ Asp Cys Met Phe
Gly Asn Gly Lys Gly Tyr⁴⁷⁰ Arg Gly Lys Arg Ala Thr Thr Val Thr Gly⁴⁸⁰ Thr Pro Cys Gln Asp Trp
Ala Ala Gln Glu⁴⁹⁰ Pro His Arg His Ser Ile Phe Thr Pro Glu⁵⁰⁰ Thr Asn Pro Arg Ala Gly Leu Glu
Lys Asn⁵¹⁰ Tyr Cys Arg Asn Pro Asp Gly Asp Val Gly⁵²⁰ Gly Pro Trp Cys Tyr Thr Thr Asn Pro
Arg⁵³⁰ Lys Leu Tyr Asp Tyr Cys Asp Val Pro Gln⁵⁴⁰ Cys Ala Ala Pro Ser Phe Asp Cys Gly Lys550
Pro Gln Val Glu Pro Lys Lys Cys Pro Gly⁵⁶⁰ Arg
<210> 5
<212> protein
<213> Homo sapiens
<400> Description of the sequence
SEQ ID NO: 5
Val Val Gly Gly Cys Val Ala His Pro⁵⁷⁰ His Ser Trp Pro Trp Gln Val Ser Leu Arg⁵⁸⁰ Thr Arg Phe
Gly Met His Phe Cys Gly Gly⁵⁹⁰ Thr Leu Ile Ser Pro Glu Trp Val Leu Thr⁶⁰⁰ Ala Ala His Cys Leu
Glu Lys Ser Pro Arg⁶¹⁰ Pro Ser Ser Tyr Lys Val Ile Leu Gly Ala⁶²⁰ His Gln Glu Val Asn Leu Glu
Pro His Val⁶³⁰ Gln Glu Ile Glu Val Ser Arg Leu Phe Leu⁶⁴⁰ Glu Pro Thr Arg Lys Asp Ile Ala Leu
Leu⁶⁵⁰ Lys Leu Ser Ser Pro Ala Val Ile Thr Asp⁶⁶⁰ Lys Val Ile Pro Ala Cys Leu Pro Set Pro⁶⁷⁰
Asn Tyr Val Val Ala Asp Arg Thr Glu Cys⁶⁸⁰ Phe Ile Thr Gly Trp Gly Glu Thr Gln Gly⁶⁹⁰ Thr Phe
Gly Ala Gly Leu Leu Lys Glu Ala⁷⁰⁰ Gln Leu Pro Val Ile Glu Asn Lys Val Cys⁷¹⁰ Asn Arg Tyr Glu
Phe Leu Asn Gly Arg Va⁷²⁰l Gln Ser Thr Glu Leu Cys Ala Gly His Leu⁷³⁰ Ala Gly Gly Thr Asp
Ser Cys Gln Gly Asp⁷⁴⁰ Ser Gly Gly Pro Leu Val Cyc Phe Glu Lys⁷⁵⁰ Asp Lys Tyr Ile Leu Gln Gly
Val Thr Ser⁷⁶⁰ Trp Gly Leu Gly Cys Ala Arg Pro Asn Lys⁷⁷⁰ Pro Gly Val Tyr Val Arg Val Ser Arg
Phe⁷⁸⁰ Val Thr Trp Ile Glu Gly Val Met Arg Asn⁷⁹⁰ Asn
<210> 6
<212> protein
<213> Homo sapiens
<400> Description of the sequence
SEQ ID NO: 6
Tyr⁸⁰ Leu Ser Glu Cys Lys Thr Gly Asn Gly Lys⁹⁰ Asn Tyr Arg Gly Thr Met Ser Lys Thr Lys¹⁰⁰
Asn Gly Ile Thr Cys Gln Lys Trp Ser Ser¹¹⁰ Thr Ser Pro His Arg Pro Arg Phe Ser Pro¹²⁰ Ala Thr
His Pro Ser Glu Gly Leu Glu Glu¹³⁰ Asn Tyr Cys Arg Asn Pro Asp Asn Asp Pro¹⁴⁰ Gln Gly Pro
Trp Cys Tyr Thr Thr Asp Pro¹⁵⁰ Glu Lys Arg Tyr Asp Tyr Cys Asp Ile Leu¹⁶⁰ Glu Cys Glu Glu Glu
Cys Met His Cys Ser¹⁷⁰ Gly Glu Asn Tyr Asp Gly Lys Ile Ser Lys¹⁸⁰ Thr Met Ser Gly Leu Glu Cys
Gln Ala Trp⁸⁰ Asp Ser Gln Ser Pro His Ala His Gly Tyr²⁰⁰ Ile Pro Ser Lys Phe Pro Asn Lys Asn
Leu²¹⁰ Lys Lys Asn Tyr Cys Arg Asn Pro Asp Arg²²⁰ Glu Leu Arg Pro Trp Cys Phe Thr Thr
Asp²³⁰ Pro Asn Lys Arg Trp Glu Leu Cys Asp Ile²⁴⁰ Pro Arg Cys Thr Thr Pro Pro Pro Ser Ser²⁵⁰
Gly Pro Thr Tyr Gln Cys Leu Lys Gly Thr²⁶⁰ Gly Glu Asn Tyr Arg Gly Asn Val Ala Val Thr Val
Ser Gly His Thr Cys Gln His Trp²⁸⁰ Ser Ala Gln Thr Pro His Thr His Asn Arg²⁹⁰ Thr Pro Glu An
Phe Pro Cys Lys Asn Leu³⁰⁰ Asp Glu Asn Tyr Cys Arg Asn Pro Asp Gly³¹⁰ Lys Arg Ala Pro Trp
Cys His Thr Thr Asn³²⁰ Ser Gln Val Arg Trp Glu Tyr Cys Lys Ile³³⁰ Pro Ser Cys Asp Ser Ser Pro
Val Ser Thr³⁴⁰ Glu Gln Leu Ala Pro Thr Ala Pro Pro Glu³⁵⁰ Leu Thr Pro Val Val Gln Asp Cys Tyr
His³⁶⁰ Gly Asp Gly Gln Ser Tyr Arg Gly Thr Ser³⁷⁰ Ser Thr Thr Thr Thr Gly Lys Lys Cys Gln³⁸⁰
Ser Trp Ser Ser Met Thr Pro His Arg His³⁹⁰ Gln Lys Thr Pro Glu Asn Tyr Pro Asn Ala⁴⁰⁰ Gly
Leu Thr Met Asn Tyr Cys Arg Asn Pro⁴¹⁰ Asp Ala Asp Lys Gly Pro Trp Cys Phe Thr⁴²⁰ Thr Asp
Pro Ser Val Arg Trp Glu Tyr Cys⁴³⁰ Asn Leu Lys Lys Cys Ser Gly Thr Glu Ala⁴⁴⁰
<210> 7
<212> protein
<213> Homo sapiens
<400> Description of the sequence
SEQ ID NO: 7
Tyr⁸⁰ Leu Ser Glu Cys Lys Thr Gly Asn Gly Lys⁹⁰ Asn Tyr Arg Gly Thr Met Ser Lys Thr Lys¹⁰⁰
Asn Gly Ile Thr Cys Gln Lys Trp Ser Ser¹¹⁰ Thr Ser Pro His Arg Pro Arg Phe Ser Pro¹²⁰ Ala Thr
His Pro Ser Glu Gly Leu Glu Glu¹³⁰ Asn Tyr Cys Arg Asn Pro Asp Asn Asp Pro¹⁴⁰ Gln Gly Pro
Trp Cys Tyr Thr Thr Asp Pro¹⁵⁰ Glu Lys Arg Tyr Asp Tyr Cys Asp Ile Leu¹⁶⁰ Glu Cys Glu Glu Glu
Cys Met His Cys Ser¹⁷⁰ Gly Glu Asn Tyr Asp Gly Lys Ile Ser Lys¹⁸⁰ Thr Met Ser Gly Leu Glu Cys
Gln Ala Trp⁸⁰ Asp Ser Gln Ser Pro His Ala His Gly Tyr²⁰⁰ Ile Pro Ser Lys Phe Pro Asn Lys Asn
Leu²¹⁰ Lys Lys Asn Tyr Cys Arg Asn Pro Asp Arg²²⁰ Glu Leu Arg Pro Trp Cys Phe Thr Thr
Asp²³⁰ Pro Asn Lys Arg Trp Glu Leu Cys Asp Ile²⁴⁰ Pro Arg Cys Thr Thr Pro Pro Pro Ser Ser²⁵⁰
Gly Pro Thr Tyr Gln Cys Leu Lys Gly Thr²⁶⁰ Gly Glu Asn Tyr Arg Gly Asn Val Ala Val Thr Val
Ser Gly His Thr Cys Gln His Trp²⁸⁰ Ser Ala Gln Thr Pro His Thr His Asn Arg²⁹⁰ Thr Pro Glu Asn
Phe Pro Cys Lys Asn Leu³⁰⁰ Asp Glu Asn Tyr Cys Arg Asn Pro Asp Gly³¹⁰ Lys Arg Ala Pro Trp
Cys His Thr Thr Asn³²⁰ Ser Gln Val Arg Trp Glu Tyr Cys Lys Ile³³⁰ Pro Ser Cys Asp Ser Ser Pro
Val
<210> 8
<212> protein
<213> Homo sapiens
<400> Description of the sequence
SEQ ID NO: 8
Tyr⁸⁰ Leu Ser Glu Cys Lys Thr Gly Asn Gly Lys⁹⁰ Asn Tyr Arg Gly Thr Met Ser Lys Thr Lys¹⁰⁰
Asn Gly Ile Thr Cys Gln Lys Trp Ser Ser¹¹⁰ Thr Ser Pro His Arg Pro Arg Phe Ser Pro¹²⁰ Ala Thr
His Pro Ser Glu Gly Leu Glu Glu¹³⁰ Asn Tyr Cys Arg Asn Pro Asp Asn Asp Pro¹⁴⁰ Gln Gly Pro
Trp Cys Tyr Thr Thr Asp Pro¹⁵⁰ Glu Lys Arg Tyr Asp Tyr Cys Asp Ile Leu¹⁶⁰ Glu Cys Glu Glu Glu
Cys Met His Cys Ser¹⁷⁰ Gly Glu Asn Tyr Asp Gly Lys Ile Ser Lys¹⁸⁰ Thr Met Ser Gly Leu Gln Cys
Gln Ala Trp⁸⁰ Asp Ser Gln Ser Pro His Ala His Gly Tyr²⁰⁰ Ile Pro Ser Lys Phe Pro Asn Lys Asn
Leu²¹⁰ Lys Lys Asn Tyr Cys Arg Asn Pro Asp Arg²²⁰ Glu Leu Arg Pro Trp Cys Phe Thr Thr
Asp²³⁰ Pro Asn Lys Arg Trp Gln Leu Cys Asp Ile²⁴⁰ Pro Arg Cys Thr Thr Pro Pro Pro Ser Ser²⁵⁰
Gly Pro Thr Tyr Gln Cys Leu Lys Gly Thr²⁶⁰ Gly Glu Asn Tyr Arg Gly Asn Val Ala Val Thr Val
Ser Gly His Thr Cys Gln His Trp²⁸⁰ Ser Ala Gln Thr Pro His Thr His Asn Arg²⁹⁰ Thr Pro Gln Asn
Phe Pro Cys Lys Asn Leu³⁰⁰ Asp Gln Asn Tyr Cys Arg Asn Pro Asp Gly³¹⁰ Lys Arg Ala Pro Trp
Cys His Thr Thr Asn³²⁰ Ser Gln Val Arg Trp Gln Tyr Cys Lys Ile³³⁰ Pro Ser Cys Asp Ser Ser Pro
Val Ser Thr³⁴⁰ Gln Gln Leu Ala Pro Thr Ala Pro Pro Glu³⁵⁰ Leu Thr Pro Val
<210> 9
<212> protein
<213> Homo sapiens
<400> Description of the sequence
SEQ ID NO: 9
Asn⁶⁰ Arg Lys Ser Ser Ile Ile Ile Arg Met Arg⁷⁰ Asp Val Val Leu Phe Glu Lys Lys⁷⁸ Val Tyr⁸⁰ Leu
Ser Glu Cys Lys Thr Gly Asn Gly Lys⁹⁰ Asn Tyr Arg Gly Thr Met Ser Lys Thr Lys¹⁰⁰ Asn Gly Ile
Thr Cys Gln Lys Trp Ser Ser¹¹⁰ Thr Ser Pro His Arg Pro Arg Phe Ser Pro¹²⁰ Ala Thr His Pro Ser
Glu Gly Leu Glu Glu¹³⁰ Asn Tyr Cys Arg Asn Pro Asp Asn Asp Pro¹⁴⁰ Gln Gly Pro Trp Cys Tyr
Thr Thr Asp Pro¹⁵⁰ Glu Lys Arg Tyr Asp Tyr Cys Asp Ile Leu¹⁶⁰ Gln Cys Gln Glu Glu Cys Met
His Cys Ser¹⁷⁰ Gly Gln Asn Tyr Asp Gly Lys Ile Ser Lys¹⁸⁰ Thr Met Ser Gly Leu Gln Cys Gln Ala
Trp⁸⁰ Asp Ser Gln Ser Pro His Ala His Gly Tyr²⁰⁰ Ile Pro Ser Lys Phe Pro Asn Lys Asn Leu²¹⁰
Lys Lys Asn Tyr Cys Arg Asn Pro Asp Arg²²⁰ Glu Leu Arg Pro Trp Cys Phe Thr Thr Asp²³⁰ Pro
Asn Lys Arg Trp Gly Leu Cys Asp Ile²⁴⁰ Pro Arg Cys Thr Thr Pro Pro Pro Ser Ser²⁵⁰ Gly Pro Thr
Tyr Gln Cys Leu Lys Gly Thr²⁶⁰ Gly Gln Asn Tyr Arg Gly Asn Val Ala Val Thr Val Ser Gly His Thr
Cys Gln His Trp²⁸⁰ Ser Aia Gln Thr Pro His Thr His Asn Arg²⁹⁰ Thr Pro Gln Asn Phe Pro Cys Lys
Asn Leu³⁰⁰ Asp Gln Asn Tyr Cys Arg Asn Pro Asp Gly³¹⁰ Lys Arg Ala Pro Trp Cys His Thr Thr
Asn³²⁰ Ser Gln Val Arg Trp Gln Tyr Cys Lys Ile³³⁰ Pro Ser Cys Asp Ser Ser Pro Val Ser Thr³⁴⁰
Gln Gln Leu Ala Pro Thr Ala Pro Pro Glu³⁵⁰ Leu Thr Pro Val Val Gln Asp Cys Tyr His³⁶⁰ Gly Asp
Gly Gln Ser Tyr Arg Gly Thr Ser³⁷⁰ Ser Thr Thr Thr Thr Gly Lys Lys Cys Gln³⁸⁰ Ser Trp Ser Ser
Met Thr Pro His Arg His³⁹⁰ Gln Lys Thr Pro Gln Asn Tyr Pro Asn Ala⁴⁰⁰ Gly Leu Thr Met Asn
Tyr Cys Arg Asn Pro⁴¹⁰ Asp Ala Asp Lys Gly Pro Trp Cys Phe Thr⁴²⁰ Thr Asp Pro Ser Val Arg
Trp Gln Tyr Cys⁴³⁰ Asn Leu Lys Lys Cys Ser Gly Thr Gln Ala⁴⁴⁰ Ser Val Val Ala Pro Pro Pro
<210> 10
<212> protein
<213> Homo sapiens
<400> Description of the sequence
SEQ ID NO: 10
Lys⁷⁸ Val Tyr⁸⁰ Leu Ser Glu Cys Lys Thr Gly Asn Gly Lys⁹⁰ Asn Tyr Arg Gly Thr Met Ser Lys Thr
Lys¹⁰⁰ Asn Gly Ile Thr Cys Gln Lys Trp Ser Ser¹¹⁰ Thr Ser Pro His Arg Pro Arg Phe Ser Pro¹²⁰
Ala Thr His Pro Ser Glu Gly Leu Glu Glu¹³⁰ Asn Tyr Cys Arg Asn Pro Asp Asn Asp Pro¹⁴⁰ Gln
Gly Pro Trp Cys Tyr Thr Thr Asp Pro¹⁵⁰ Glu Lys Arg Tyr Asp Tyr Cys Asp Ile Leu¹⁶⁰ Glu Cys Glu
Glu Glu Cys Met His Cys Ser¹⁷⁰ Gly Glu Asn Tyr Asp Gly Lys Ile Ser Lys¹⁸⁰ Thr Met Ser Gly Leu
Glu Cys Gln Ala Trp⁸⁰ Asp Ser Gln Ser Pro His Ala His Gly Tyr²⁰⁰ Ile Pro Ser Lys Phe Pro Asn
Lys Asn Leu²¹⁰ Lys Lys Asn Tyr Cys Arg Asn Pro Asp Arg²²⁰ Glu Leu Arg Pro Trp Cys Phe Thr
Thr Asp²³⁰ Pro Asn Lys Arg Trp Glu Leu Cys Asp Ile²⁴⁰ Pro Arg Cys Thr Thr Pro Pro Pro Ser
Ser²⁵⁰ Gly Pro Thr Tyr Gln Cys Leu Lys Gly Thr²⁶⁰ Gly Glu Asn Tyr Arg Gly Asn Val Ala Val Thr
Val Ser Gly His Thr Cys Gln His Trp²⁸⁰ Ser Ala Gln Thr Pro His Thr His Asn Arg²⁹⁰ Thr Pro Glu
Asn Phe Pro Cys Lys Asn Leu³⁰⁰ Asp Glu Asn Tyr Cys Arg Asn Pro Asp Gly³¹⁰ Lys Arg Ala Pro
Trp Cys His Thr Thr Asn³²⁰ Ser Gln Val Arg Trp Glu Tyr Cys Lys Ile³³⁰ Pro Ser Cys Asp Ser Ser
Pro Val Ser Thr³⁴⁰ Glu Gln Leu Ala Pro Thr Ala Pro Pro Glu³⁵⁰ Leu Thr Pro Val Val Gln Asp Cys
Tyr His³⁶⁰ Gly Asp Gly Gln Ser Tyr Arg Gly Thr Ser³⁷⁰ Ser Thr Thr Thr Gly Lys Lys Cys
Gln³⁸⁰ Ser Trp Ser Ser Met Thr Pro His Arg His³⁹⁰ Gln Lys Thr Pro Glu Asn Tyr Pro Asn Ala⁴⁰⁰
Gly Leu Thr Met Asn Tyr Cys Arg Asn Pro⁴¹⁰ Asp Ala Asp Lys Gly Pro Trp Cys Phe Thr⁴²⁰ Thr
Asp Pro Ser Val Arg Trp Glu Tyr Cys⁴³⁰ Asn Leu Lys Lys Cys Ser Gly Thr Glu Ala⁴⁴⁰ Ser Val Val
Ala Pro Pro Pro
<210> 11
<212> protein
<213> Homo sapiens
<400> Description of the sequence
SEQ ID NO: 11
Lys⁷⁸ Val Tyr⁸⁰ Leu Ser Glu Cys Lys Thr Gly Asn Gly Lys⁹⁰ Asn Tyr Arg Gly Thr Met Ser Lys Thr
Lys¹⁰⁰ Asn Gly Ile Thr Cys Gln Lys Trp Ser Ser¹¹⁰ Thr Ser Pro His Arg Pro Arg Phe Ser Pro¹²⁰
Ala Thr His Pro Ser Glu Gly Leu Glu Glu¹³⁰ Asn Tyr Cys Arg Asn Pro Asp Asn Asp Pro¹⁴⁰ Gln
Gly Pro Trp Cys Tyr Thr Thr Asp Pro¹⁵⁰ Glu Lys Arg Tyr Asp Tyr Cys Asp Ile Leu¹⁶⁰ Glu Cys Glu
Glu Glu Cys Met His Cys Ser¹⁷⁰ Gly Glu Asn Tyr Asp Gly Lys Ile Ser Lys¹⁸⁰ Thr Met Ser Gly Leu
Glu Cys Gln Ala Trp⁸⁰ Asp Ser Gln Ser Pro His Ala His Gly Tyr²⁰⁰ Ile Pro Ser Lys Phe Pro Asn
Lys Asn Leu²¹⁰ Lys Lys Asn Tyr Cys Arg Asn Pro Asp Arg²²⁰ Glu Leu Arg Pro Trp Cys Phe Thr
Thr Asp²³⁰ Pro Asn Lys Arg Trp Glu Leu Cys Asp Ile²⁴⁰ Pro Arg Cys Thr Thr Pro Pro Pro Ser
Ser Gly Pro Thr Tyr Gln Cys Leu Lys Gly Thr²⁶⁰ Gly Glu Asn Tyr Arg Gly Asn Val Ala Val Thr
Val Ser Gly His Thr Cys Gln His Trp²⁸⁰ Ser Ala Gln Thr Pro His Thr His Asn Arg²⁹⁰ Thr Pro Glu
Asn Phe Pro Cys Lys Asn Leu³⁰⁰ Asp Glu Asn Tyr Cys Arg Asn Pro Asp Gly³¹⁰ Lys Arg Ala Pro
Trp Cys His Thr Thr Asn³²⁰ Ser Gln Val Arg Trp Glu Tyr Cys Lys Ile³³⁰ Pro Ser Cys Asp Ser Ser
Pro Val Ser Thr³⁴⁰ Glu Gln Leu Ala Pro Thr Ala Pro Pro Glu³⁵⁰ Leu Thr Pro Val Val Gln Asp Cys
Tyr His³⁶⁰ Gly Asp Gly Gln Ser Tyr Arg Gly Thr Ser³⁷⁰ Ser Thr Thr Thr Thr Gly Lys Lys Cys
Gln³⁸⁰ Ser Trp Ser Ser Met Thr Pro His Arg His³⁹⁰ Gln Lys Thr Pro GIU Asn Tyr Pro Asn Ala⁴⁰⁰
Gly Leu Thr Met Asn Tyr Cys Arg Asn Pro⁴¹⁰ Asp Ala Asp Lys Gly Pro Trp Cys Phe Thr⁴²⁰ Thr
Asp Pro Ser Val Arg Trp Glu Tyr Cys⁴³⁰ Asn Leu Lys Lys Cys Ser Gly Thr Glu Ala⁴⁴⁰ Ser Val Val
Ala Pro Pro
<210> 12
<212> protein
<213> Homo sapiens
<400> Description of the sequence
SEQ ID NO: 12
Lys⁷⁸ Val Tyr⁸⁰ Leu Set Glu Cys Lys Thr Gly Asn Gly Lys⁹⁰ Asn Tyr Arg Gly Thr Met Ser Lys Thr
Lys¹⁰⁰ Asn Gly Ile Thr Cys Gln Lys Trp Ser Ser¹¹⁰ Thr Ser Pro His Arg Pro Arg Phe Ser Pro¹²⁰
Ala Thr His Pro Set Glu Gly Leu Glu Glu¹³⁰ Asn Tyr Cys Arg Asn Pro Asp Asn Asp Pro¹⁴⁰ Gln
Gly Pro Trp Cys Tyr Thr Thr Asp Pro¹⁵⁰ Glu Lys Arg Tyr Asp Tyr Cys Asp Ile Leu¹⁶⁰ Glu Cys Glu
Glu Glu Cys Met His Cys Ser¹⁷⁰ Gly Glu Asn Tyr Asp Gly Lys Ile Ser Lys¹⁸⁰ Thr Met Ser Gly Leu
Glu Cys Gln Ala Trp⁸⁰ Asp Ser Gln Ser Pro His Ala His Gly Tyr²⁰⁰ Ile Pro Ser Lys Phe Pro Asn
Lys Asn Leu²¹⁰ Lys Lys Asn Tyr Cys Arg Asn Pro Asp Arg²²⁰ Glu Leu Arg Pro Trp Cys Phe Thr
Thr Asp²³⁰ Pro Asn Lys Arg Trp Glu Leu Cys Asp Ile²⁴⁰ Pro Arg Cys Thr Thr Pro Pro Pro Ser
Ser²⁵⁰ Gly Pro Thr Tyr Gln Cys Leu Lys Gly Thr²⁶⁰ Gly Glu Asn Tyr Arg Gly Asn Val Ala Val Thr
Val Ser Gly His Thr Cys Gln His Trp²⁸⁰ Ser Ala Gln Thr Pro His Thr His Asn Arg²⁹⁰ Thr Pro Glu
Asn Phe Pro Cys Lys Asn Leu³⁰⁰ Asp Glu Asn Tyr Cys Arg Asn Pro Asp Gly³¹⁰ Lys Arg Ala Pro
Trp Cys His Thr Thr Asn³²⁰ Set Gln Val Arg Trp Glu Tyr Cys Lys Ile³³⁰ Pro Ser Cys Asp Ser Ser
Pro Val Ser Thr³⁴⁰ Glu Gln Leu Ala Pro Thr Ala Pro Pro Glu³⁵⁰ Leu Thr Pro Val Val Gln Asp Cys
Tyr His³⁶⁰ Gly Asp Gly Gln Ser Tyr Arg Gly Thr Ser³⁷⁰ Ser Thr Thr Thr Thr Gly Lys Lys Cys
Gln³⁸⁰ Ser Trp Ser Ser Met Thr Pro His Arg His³⁹⁰ Gln Lys Thr Pro Glu Asn Tyr Pro Asn Ala⁴⁰⁰
Gly Leu Thr Met Asn Tyr Cys Arg Asn Pro⁴¹⁰ Asp Ala Asp Lys Gly Pro Trp Cys Phe Thr⁴²⁰ Thr
Asp Pro Ser Val Arg Trp Glu Tyr Cys⁴³⁰ Asn Leu Lys Lys Cys Set Gly Thr Glu Ala⁴⁴⁰ Ser Val Val
Ala Pro Pro Pro Val Val Leu⁴⁵⁰ Leu Pro Asp Val Glu Thr Pro Ser Glu Glu⁴⁶⁰ Asp Cys Met Phe
Gly Asn Gly Lys
<210> 13
<212> protein
<213> Homo sapiens
<400> Description of the sequence
SEQ ID NO: 13
Lys⁷⁸ Val Tyr⁸⁰ Leu Ser Glu Cys Lys Thr Gly Asn Gly Lys⁹⁰ Asn Tyr Arg Gly Thr Met Ser Lys Thr
Ly^{s100} Asn Gly Ile Thr Cys Gln Lys Trp Ser Ser¹¹⁰ Thr Ser Pro His Arg Pro Arg Phe Ser Pro¹²⁰
Ala Thr His Pro Ser Glu Gly Leu Glu Glu¹³⁰ Asn Tyr Cys Arg Asn Pro Asp Asn Asp Pro¹⁴⁰ Gln
Gly Pro Trp Cys Tyr Thr Thr Asp Pro¹⁵⁰ Glu Lys Arg Tyr Asp Tyr Cys Asp Ile Leu¹⁶⁰ Glu Cys Glu
Glu Glu Cys Met His Cys Ser¹⁷⁰ Gly Glu Asn Tyr Asp Gly Lys Ile Ser Lys¹⁸⁰ Thr Met Ser Gly Leu
Glu Cys Gln Ala Trp⁸⁰ Asp Ser Gln Ser Pro His Ala His Gly Tyr²⁰⁰ Ile Pro Ser Lys Phe Pro Asn
Lys Asn Leu²¹⁰ Lys Lys Asn Tyr Cys Arg Asn Pro Asp Arg²²⁰ Glu Leu Arg Pro Trp Cys Phe Thr
Thr Asp²³⁰ Pro Asn Lys Arg Trp Glu Leu Cys Asp Ile²⁴⁰ Pro Arg Cys Thr Thr Pro Pro Pro Ser
Ser²⁵⁰ Gly Pro Thr Tyr Gln Cys Leu Lys Gly Thr²⁶⁰ Gly Glu Asn Tyr Arg Gly Asn Val Ala Val Thr
Val Ser Gly His Thr Cys Gln His Tr^{p280} Ser Ala Gln Thr Pro His Thr His Asn Arg²⁹⁰ Thr Pro Glu
Asn Phe Pro Cys Lys Asn Leu³⁰⁰ Asp Glu Asn Tyr Cys Arg Asn Pro Asp Gly³¹⁰ Lys Arg Ala Pro
Trp Cys His Thr Thr Asn³²⁰ Ser Gln Val Arg Trp Glu Tyr Cys Lys Ile³³⁰ Pro Ser Cys Asp Ser Ser
Pro Val Ser Thr³⁴⁰ Glu Gln Leu Ala Pro Thr Ala Pro Pro Glu³⁵⁰ Leu Thr Pro Val Val Gln Asp Cys
Tyr His³⁶⁰ Gly Asp Gly Gln Ser Tyr Arg Gly Thr Ser³⁷⁰ Ser Thr Thr Thr Thr Gly Lys Lys Cys
Gln³⁸⁰ Ser Trp Ser Ser Met Thr Pro His Arg His³⁹⁰ Gln Lys Thr Pro Glu Asn Tyr Pro Asn Ale⁴⁰⁰
Gly Leu Thr Met Asn Tyr Cys Arg Asn Pro⁴¹⁰ Asp Ala Asp Lys Gly Pro Trp Cys Phe Thr⁴²⁰ Thr
Asp Pro Ser Val Arg Trp Glu Tyr Cys⁴³⁰ Asn Leu Lys Lys Cys Ser Gly Thr Glu Ala⁴⁴⁰ Ser Val Val
Ala Pro Pro Pro Val Val Leu⁴⁵⁰ Leu Pro Asp Val Glu Thr Pro Ser Glu Glu⁴⁶⁰ Asp Cys Met Phe
Gly Asn Gly Lys Gly Tyr⁴⁷⁰ Arg Gly Lys Arg Ala Thr Thr Val Thr Gly⁴⁸⁰ Thr Pro Cys Gln Asp Trp
Ala Ala Gln Glu⁴⁹⁰ Pro His Arg His Ser Ile Phe Thr Pro Glu⁵⁰⁰ Thr Asn Pro Arg Ala Gly Leu Glu
Lys Asn⁵¹⁰ Tyr Cys Arg Asn Pro Asp Gly Asp Val Gly⁵²⁰ Gly Pro Trp Cys Tyr Thr Thr Asn Pro
Arg⁵³⁰
<210> 14
<212> protein
<213> Homo sapiens
<400> Description of the sequence
SEQ ID NO: 14
Val Gln Asp Cys Tyr His³⁶⁰ Gly Asp Gly Gln Ser Tyr Arg Gly Thr Ser³⁷⁰ Ser Thr Thr Thr Thr Gly
Lys Lys Cys Gln³⁸⁰ Ser Trp Ser Ser Met Thr Pro His Arg His³⁹⁰ Gln Lys Thr Pro Glu Asn Tyr Pro
Asn Ala⁴⁰⁰ Gly Leu Thr Met Asn Tyr Cys Arg Asn Pro⁴¹⁰ Asp Ala Asp Lys Gly Pro Trp Cys Phe
Thr⁴²⁰ Thr Asp Pro Ser Val Arg Trp Glu Tyr Cys⁴³⁰ Asn Leu Lys Lys Cys Ser Gly Thr Glu Ala⁴⁴⁰
Ser Val Val Ala Pro Pro Pro Val Val Leu⁴⁵⁰ Leu Pro Asp Val Glu Thr Pro Ser Glu Glu⁴⁶⁰ Asp Cys
Met Phe Gly Asn Gly Lys Gly Tyr⁴⁷⁰ Arg Gly Lys Arg Ala Thr Thr Val Thr Gly⁴⁸⁰ Thr Pro Cys Gln
Asp Trp Ala Ala Gln Glu⁴⁹⁰ Pro His Arg His Ser Ile Phe Thr Pro Glu⁵⁰⁰ Thr Asn Pro Arg Ala Gly
Leu Glu Lys Asn⁵¹⁰ Tyr Cys Arg Asn Pro Asp Gly Asp Val Gly⁵²⁰ Gly Pro Trp Cys Tyr Thr Thr
Asn Pro Arg⁵³⁰ Lys Leu Tyr Asp Tyr Cys Asp Val Pro Gln⁵⁴⁰ Cys Ala Ala Pro Ser Phe
<210> 15
<212> protein
<213> Homo sapiens
<400> Description of the sequence
SEQ ID NO: 15
Tyr⁸⁰ Leu Ser Glu Cys Lys Thr Gly Asn Gly Lys⁹⁰ Asn Tyr Arg Gly Thr Met Ser Lys Thr Lys¹⁰⁰
Asn Gly Ile Thr Cys Gln Lys Trp Ser Ser¹¹⁰ Thr Ser Pro His Arg Pro Arg Phe Ser Pro¹²⁰ Ala Thr
His Pro Ser Glu Gly Leu Glu Glu¹³⁰ Asn Tyr Cys Arg Asn Pro Asp Asn Asp Pro¹⁴⁰ Gln Gly Pro
Trp Cys Tyr Thr Thr Asp Pro¹⁵⁰ Glu Lys Arg Tyr Asp Tyr Cys Asp Ile Leu¹⁶⁰ Glu Cys Glu Glu
<210> 16
<212> protein
<213> Homo sapiens
<400> Description of the sequence
SEQ ID NO: 16
Cys Met His Cys Ser¹⁷⁰ Gly Glu Asn Tyr Asp Gly Lys Ile Ser Lys¹⁸⁰ Thr Met Ser Gly Leu Glu Cys
Gln Ala Trp⁸⁰ Asp Ser Gln Ser Pro His Ala His Gly Tyr²⁰⁰ Ile Pro Ser Lys Phe Pro Asn Lys Asn
Leu Lys Lys Asn Tyr Cys Arg Asn Pro Asp Arg²²⁰ Glu Leu Arg Pro Trp Cys Phe Thr Thr
Asp²³⁰ Pro Asn Lys Arg Trp Glu Leu Cys Asp Ile²⁴⁰ Pro Arg Cys Thr Thr Pro Pro Pro Ser Ser²⁵⁰
Gly Pro Thr Tyr Gln Cys Leu Lys Gly Thr²⁶⁰ Gly Glu Asn Tyr Arg Gly Asn Val Ala Val Thr Val
Ser Gly His Thr Cys Gln His Trp²⁸⁰ Ser Ala Gln Thr Pro His Thr His Asn Arg²⁹⁰ Thr Pro Glu Asn
Phe Pro Cys Lys Asn Leu³⁰⁰ Asp Glu Asn Tyr Cys Arg Asn Pro Asp Gly³¹⁰ Lys Arg Ala Pro Trp
Cys His Thr Thr Asn³²⁰ Ser Gln Val Arg Trp Glu Tyr Cys Lys Ile³³⁰ Pro Ser Cys Asp Ser Ser Pro
Val
<210> 17
<212> protein
<213> Homo sapiens
<400> Description of the sequence
SEQ ID NO: 17
Val Gln Asp Cys Tyr His³⁶⁰ Gly Asp Gly Gln Ser Tyr Arg Gly Thr Ser³⁷⁰ Ser Thr Thr Thr Thr Gly
Lys Lys Cys Gln³⁸⁰ Ser Trp Ser Ser Met Thr Pro His Arg His³⁹⁰ Gln Lys Thr Pro Glu Asn Tyr Pro
Asn Ala⁴⁰⁰ Gly Leu Thr Met Asn Tyr Cys Arg Asn Pro⁴¹⁰ Asp Ala Asp Lys Gly Pro Trp Cys Phe
Thr⁴²⁰ Thr Asp Pro Ser Val Arg Trp Glu Tyr Cys⁴³⁰ Asn Leu Lys Lys Cys Ser Gly Thr Glu Ala⁴⁴⁰
<210> 18
<212> protein
<213> Homo sapiens
<400> Description of the sequence
SEQ ID NO: 18
Ser Val Val Ala Pro Pro Pro Val Val Leu⁴⁵⁰ Leu Pro Asp Val Glu Thr Pro Ser Glu Glu⁴⁶⁰ Asp Cys
Met Phe Gly Asn Gly Lys Gly Tyr⁴⁷⁰ Arg Gly Lys Arg Ala Thr Thr Val Thr Gly⁴⁸⁰ Thr Pro Cys Gln
Asp Trp Ala Ala Gln Glu⁴⁹⁰ Pro His Arg His Ser Ile Phe Thr Pro Glu⁵⁰⁰ Thr Asn Pro Arg Ala Gly
Leu Glu Lys Asn⁵¹⁰ Tyr Cys Arg Asn Pro Asp Gly Asp Val Gly⁵²⁰ Gly Pro Trp Cys Tyr Thr Thr
Asn Pro Arg⁵³⁰ Lys Leu Tyr Asp Tyr Cys Asp Val Pro Gln⁵⁴⁰ Cys Ala Ala Pro Ser Phe
<210> 19
<212> protein
<213> Homo sapiens
<400> Description of the sequence
SEQ ID NO: 19
Val Val Ala Pro Pro Pro Val Val Leu⁴⁵⁰ Leu Pro Asp Val Glu Thr Pro Ser Glu Glu⁴⁶⁰ Asp Cys
Met Phe Gly Asn Gly Lys Gly Tyr⁴⁷⁰ Arg Gly Lys Arg Ala Thr Thr Val Thr Gly⁴⁸⁰ Thr Pro Cys Gln
Asp Trp Ala Ala Gln Glu⁴⁹⁰ Pro His Arg His Ser Ile Phe Thr Pro Glu⁵⁰⁰ Thr Asn Pro Arg Ala Gly
Leu Glu Lys Asn⁵¹⁰ Tyr Cys Arg Asn Pro Asp Gly Asp Val Gly⁵²⁰ Gly Pro Trp Cys Tyr Thr Thr
Asn Pro Arg⁵³⁰ Lys Leu Tyr Asp Tyr Cys Asp Val Pro Gln⁵⁴⁰ Cys Ala Ala Pro Ser Phe Asp Cys
Gly Lys⁵⁵⁰ Pro Gln Val Glu Pro Lys Lys Cys Pro Gly⁵⁶⁰ Arg
<210> 20
<212> 6enoκ
<213> Homo sapiens
<400> Description of the sequence
SEQ ID NO: 20
Val Val Ala Pro Pro Pro Val Val Leu⁴⁵⁰ Leu Pro Asp Val Glu Thr Pro Ser Glu Glu⁴⁶⁰ Asp Cys
Met Phe Gly Asn Gly Lys Gly Tyr⁴⁷⁰ Arg Gly Lys Arg Ala Thr Thr Val Thr Gly⁴⁸⁰ Thr Pro Cys Gln
Asp Trp Ala Ala Gln Glu⁴⁹⁰ Pro His Arg His Ser Ile Phe Thr Pro Glu⁵⁰⁰ Thr Asn Pro Arg Ala Gly
Leu Glu Lys Asn⁵¹⁰ Tyr Cys Arg Asn Pro Asp Gly Asp Val Gly⁵²⁰ Gly Pro Trp Cys Tyr Thr Thr
Asn Pro Arg⁵³⁰ Lys Leu Tyr Asp Tyr Cys Asp Val Pro Gln⁵⁴⁰ Cys Ala Ala Pro Ser Phe Asp Cys
Gly Lys⁵⁵⁰ Pro Gln Val Glu Pro Lys Lys Cys Pro Gly⁵⁶⁰ Arg Val Val Gly Gly Cys Val Ala His
Pro⁵⁷⁰ His Ser Trp Pro Trp Gln Val Ser Leu Arg⁵⁸⁰ Thr Arg Phe Gly Met His Phe Cys Gly Gly⁵⁹⁰
Thr Leu Ile Ser Pro Glu Trp Val Leu Thr⁶⁰⁰ Ala Ala His Cys Leu Glu Lys Ser Pro Arg⁶¹⁰ Pro Ser
Ser Tyr Lys Val Ile Leu Gly Ala⁶²⁰ His Gln Glu Val Asn Leu Glu Pro His Val⁶³⁰ Gln Glu Ile Glu
Val Ser Arg Leu Phe Leu⁶⁴⁰ Glu Pro Thr Arg Lys Asp Ile Ala Leu Leu⁶⁵⁰ Lys Leu Ser Ser Pro Ala
Val Ile Thr Asp⁶⁶⁰ Lys Val Ile Pro Ala Cys Leu Pro Ser Pro⁶⁷⁰ Asn Tyr Val Val Ala Asp Arg Thr
Glu Cys⁶⁸⁰ Phe Ile Thr Gly Trp Gly Glu Thr Gln Gly⁶⁹⁰ Thr Phe Gly Ala Gly Leu Leu Lys Glu
Ala⁷⁰⁰ Gln Leu Pro Val Ile Glu Asn Lys Val Cys⁷¹⁰ Asn Arg Tyr Glu Phe Leu Asn Gly Arg Va⁷²⁰l
Gln Ser Thr Glu Leu Cys Ala Gly His Leu⁷³⁰ Ala Gly Gly Thr Asp Ser Cys Gln Gly Asp⁷⁴⁰ Ser
Gly Gly Pro Leu Val Cys Phe Glu Lys⁷⁵⁰ Asp Lys Tyr He Leu Gln Gly Val Thr Ser⁷⁶⁰ Trp Gly Leu
Gly Cys Ala Arg Pro Asn Lys⁷⁷⁰ Pro Gly Val Tyr Val Arg Val Ser Arg Phe⁷⁸⁰ Val Thr Trp Ile Glu
Gly Val Met Arg Asn⁷⁹⁰ Asn

### INDUSTRIAL APPLICABILITY

The present invention is embodied with multipurpose equipment extensively employed by the industry.

1. A method of identifying a subject suffering from cancer, by identifying autoantibodies to human plasminogen and/or its fragments in a plasma of said subject.

2. The method of claim 1, wherein an indicator of a presence of cancer in the subject is an excess of the autoantibodies level by more than 20% over a reference sample of autoantibodies.

3. The method of claim 1, wherein the identification of the autoantibodies is carried out by ELISA using specifically interacting these autoantibodies with antigens mentioned above.

4. The method of claim 3, wherein the antigen for identification of the autoantibodies is a full-size or containing human plasminogen kringle fragment of human plasminogen.

5. The method of claim 4, wherein the human plasminogen fragments to identify autoantibodies selected from a list: Lys-plasminogen, the heavy chain (Glu-H), heavy chain (Lys-H), light chain (L), K1-4 (Tyr80-Ala440), K1-3 (Tyr80-Val338), K1-3 (Tyr80-Val354), K1-4 (Asn60-Pro447), K1-4 (Lys78-Pro447), K1-4 (Lys78-Pro446), K1-4 (Lys78-Lys468), K1-4, 5 (Lys78-Arg530), C4-5 (Val355-Phe546), K1 (Tur80-Glu164), K2-3 (Cys165-Val338), K4 (Val354-Ala440), K5 (Ser441-Fhe546), K5 (Val442-Arg561), mini-plasmin.

6. A diagnostic ELISA system for detection of autoantibodies to human plasminogen and/or fragments thereof, comprising: a solid carrier and an antigen, capable of specifically binding to these autoantibodies.

7. The diagnostic system according to claim 6, wherein the antigen is a full molecule of human plasminogen or its fragment having at least one kringle.

8. The diagnostic system of claim 7, wherein the human plasminogen fragment is selected from a list: Glu-plasminogen, miniplasmin, Lys-plasminogen, the heavy chain (Glu-H), heavy chain (Lys-H), light chain (L), K1- 4 (Tyr80-Ala440), K1-3 (Tyr80-Val338), K1-3 (Tyr80-Val354), K1-4 (Asn60-Pro447), K1-4 (Lys78-Pro447), K1-4 (Lys78-Pro446), K1-4 (Lys78-Lys468), K1-4, 5 (Lys78-Arg530), C4-5 (Val355-Phe546), K1 (Tur80-Glu164), K2-3 (Cys165-Val338), K4 (Val354-Ala440), K5 (Ser441-Fhe546), K5 (Val442-Arg561).


SEQUENCE LISTING (20 sequences)

SEQ ID NO: 1 - protein, 791 aa, Homo sapiens
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 2 - protein, 714 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 3 - protein, 561 aa, Homo sapiens
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGR

SEQ ID NO: 4 - protein, 484 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGR

SEQ ID NO: 5 - protein, 230 aa, Homo sapiens
VVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 6 - protein, 361 aa, Homo sapiens
YLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEA

SEQ ID NO: 7 - protein, 259 aa, Homo sapiens
YLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPV

SEQ ID NO: 8 - protein, 275 aa, Homo sapiens
YLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPV

SEQ ID NO: 9 - protein, 388 aa, Homo sapiens
NRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPP

SEQ ID NO: 10 - protein, 370 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPP

SEQ ID NO: 11 - protein, 369 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPP

SEQ ID NO: 12 - protein, 391 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGK

SEQ ID NO: 13 - protein, 453 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPR

SEQ ID NO: 14 - protein, 192 aa, Homo sapiens
VQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSF

SEQ ID NO: 15 - protein, 85 aa, Homo sapiens
YLSECKTGNGKNYRGTMSKTKNGITCQKWSSTSPHRPRFSPATHPSEGLEENYCRNPDNDPQGPWCYTTDPEKRYDYCDILECEE

SEQ ID NO: 16 - protein, 173 aa, Homo sapiens
CMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPV

SEQ ID NO: 17 - protein, 86 aa, Homo sapiens
VQDCYHGDGQSYRGTSSTTTTGKKCQSWSSMTPHRHQKTPENYPNAGLTMNYCRNPDADKGPWCFTTDPSVRWEYCNLKKCSGTEA

SEQ ID NO: 18 - protein, 106 aa, Homo sapiens
SVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSF

SEQ ID NO: 19 - protein, 120 aa, Homo sapiens
VVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGR

SEQ ID NO: 20 - protein, 350 aa, Homo sapiens
VVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN